(19) United States
(12) Patent Application Publication
(10) Pub. No.: US 2025/0248956 A1
(43) Pub. Date: Aug. 7, 2025

(54) LIQUID-DISPERSIBLE HALOPYRUVATE FORMULATIONS AND ASSOCIATED METHODS

(71) Applicant: Young Hee KO, Baltimore, MD (US)

(72) Inventor: Young Hee KO, Baltimore, MD (US)

(21) Appl. No.: 18/854,763

(22) PCT Filed: Apr. 6, 2023
(86) PCT No.: PCT/US2023/017800

§ 371 Date: Oct. 7, 2024

Related U.S. Application Data
(60) Provisional application No. 63/328,197, filed on Apr. 6, 2022.

(51) Int. Cl. A61K 31/19; A61K 9/20; A61K 31/137; A61K 31/35; A61K 31/366; A61K 38/45; A61K 45/06

(52) U.S. Cl. CPC A61K 31/19; A61K 9/2018; A61K 9/2081; A61K 31/137; A61K 31/35; A61K 31/366; A61K 38/45; A61K 45/06; C12Y 207/01001

(57) ABSTRACT

An anti-cancer formulation is described including a dry liquid dispersible composition of a cellular energy inhibitor admixed with a reactive ingredient and a pharmaceutically acceptable carrier, wherein the cellular energy inhibitor has a structure according to formula I; wherein R is selected from one of OR′, N(R″)₂, C(O)R″′, C1-C6 alkyl, C6-C12 aryl, C1-C6 heteroalkyl, C6-C12 heteroaryl, H, or an alkali metal, where R′ is selected from one of H, an alkali metal, C1-C6 alkyl, C6-C12 aryl or C(O)R″′, where R″ is selected from one of H, C1-C6 alkyl, or C6-C12 aryl, and where R″′ is selected from on of H, C1-C20 alkyl or C6-C12 aryl.

### CROSS-REFERENCE TO RELATED APPLICATIONS

This application claims the benefit of U.S. Provisional Patent Application No. 63/328,197, filed on Apr. 6, 2022, which is incorporated herein by reference in its entirety.

### BACKGROUND

Pharmaceutical formulations have a wide variety of physical forms and compositional formulations, depending on the active agent in the formulation, the route of administration, etc. For example, a solid pharmaceutical formulation includes an active agent dispersed in a solid pharmaceutical carrier. Similarly, a liquid pharmaceutical formulation includes an active agent dispersed in a liquid pharmaceutical carrier. Additional additives can vary depending on whether the dosage form is a liquid or solid, for example. General categories include diluents, disintegrants, binding agents, adhesives, wetting agents, lubricants, glidants, dyes, flavoring agents, to name a few.

### BRIEF DESCRIPTION OF THE DRAWINGS

FIG. 1 illustrates a dry liquid dispersible formulation in accordance with an example embodiment;

FIG. 2 illustrates a dry liquid dispersible formulation in accordance with an example embodiment;

FIG. 3 illustrates a dry liquid dispersible formulation in accordance with an example embodiment;

FIG. 4 illustrates a dry liquid dispersible formulation in accordance with an example embodiment;

FIG. 5 illustrates a dry liquid dispersible formulation in accordance with an example embodiment;

FIG. 6 illustrates a dry liquid dispersible formulation in accordance with an example embodiment;

FIG. 7 illustrates a dry liquid dispersible formulation in accordance with an example embodiment;

FIG. 8A illustrates a dry liquid dispersible formulation in accordance with an example embodiment;

FIG. 8B illustrates a dry liquid dispersible formulation in accordance with an example embodiment;

FIG. 9 illustrates a dry liquid dispersible formulation in accordance with an example embodiment;

FIG. 10 illustrates a dry liquid dispersible formulation in accordance with an example embodiment; and

FIG. 11 illustrates stability data by storage temperature over time of a dry liquid dispersible formulation in accordance with an example embodiment.

### DESCRIPTION OF EMBODIMENTS

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only, and is not intended to be limiting. Unless defined otherwise, all technical and scientific terms used herein have the same meaning as commonly understood by one of ordinary skill in the art to which this disclosure belongs. Also, the same reference numerals in appearing in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, network examples, etc., to provide a thorough understanding of various embodiments. One skilled in the relevant art will recognize, however, that such detailed embodiments do not limit the overall concepts articulated herein, but are merely representative thereof. One skilled in the relevant art will also recognize that the technology can be practiced without one or more of the specific details, or with other methods, components, layouts, etc. In other instances, well-known structures, materials, or operations may not be shown or described in detail to avoid obscuring aspects of the disclosure.

In this application, “comprises,” “comprising,” “containing” and “having” and the like can have the meaning ascribed to them in U.S. patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. patent law. “Consisting essentially of” or “consists essentially of” have the meaning generally ascribed to them by U.S. patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term in this written description, like “comprising” or “including,” it is understood that direct support should be afforded also to “consisting essentially of” language as well as “consisting of” language as if stated explicitly and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a given term, metric, value, range endpoint, or the like. The degree of flexibility for a particular variable can be readily determined by one skilled in the art. However, unless otherwise expressed, the term “about” generally provides flexibility of less than 0.01%. It is to be understood that, even when the term “about” is used in the present specification in connection with a specific numerical value, support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of phrases including “an example” or “an embodiment” in various places throughout this specification are not necessarily all referring to the same example or embodiment.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The formulations of the present invention may include a pharmaceutically acceptable carrier and other ingredients as dictated by the particular needs of the specific dosage formulation. Such ingredients are well known to those skilled in the art. See for example, Gennaro, A. Remington: The Science and Practice of Pharmacy 19^{th} ed. (1995), which is incorporated by reference in its entirety.

As used herein, “subject” refers to a mammal that may benefit from the administration of a drug composition or method of this invention. Examples of subjects include humans, and other animals such as horses, pigs, cattle, sheep, goats, dogs (felines), cats (canines), rabbits, rodents, primates, and aquatic mammals. In one embodiment, subject can refer to a human.

As used herein, “drug,” “active agent,” “bioactive agent,” “pharmaceutically active agent,” “therapeutically active agent” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical and medicinal arts. Further, when these terms are used, or when a particular active agent is specifically identified by name or category, it is understood that such recitation is intended to include the active agent per se, as well as pharmaceutically acceptable salts, or compounds significantly related thereto, including without limitation, prodrugs, active metabolites, isomers, and the like. The terms “cellular energy inhibitor,” “glycolysis inhibitor,” “mitochondrial inhibitor,” and the like, are considered to be active agents.

As used herein, the terms “inhibit,” “inhibiting,” or any other derivative thereof refers to the process of holding back, suppressing or restraining so as to block, prevent, limit, or decrease a rate of action or function. The use of the term is not to be misconstrued to be only of absolute prevention but can be a referent to any minute incremental step of limiting or reducing a function through the full and absolute prevention of the function.

As used herein, “cellular energy inhibitor” refers to a compound that inhibits ATP production in a cell. In some examples, a cellular energy inhibitor can inhibit glycolysis, oxidative phosphorylation, or both glycolysis and oxidative phosphorylation in a cell.

As used herein, “glycolysis inhibitor” refers to a compound that inhibits, reduces, or stops, glycolysis in a cell.

As used herein, “mitochondria inhibitor” refers to a compound that inhibits, reduces, or stops mitochondrial production of ATP in a cell.

As used herein, the terms “dosage form,”, “formulation” and “composition” are used interchangeably and refer to a mixture of two or more compounds, elements, or molecules. In some examples, the terms “dosage form,” “formulation,” and “composition” may be used to refer to a mixture of one or more active agents with a carrier and/or other excipient.

As used herein, “carrier” or “pharmaceutically acceptable carrier” refers to a substance with which a drug may be combined to achieve a specific dosage formulation for delivery to a subject. In some examples, a carrier may or may not enhance drug delivery. As a general principle, carriers do not react with the drug in a manner that substantially degrades or otherwise adversely affects the drug, except that some carriers may react with a drug to prevent it from exerting a therapeutic effect until the drug is released from the carrier. Further, the carrier, or at least a portion thereof must be physiologically suitable for administration into a subject along with the drug.

The term “excipient” herein includes any substance used, for example, as a carrier for an active agent in a liquid formulation, any substance added to the active agent and/or a solid formulation to, for example, improve its handling properties, permit the resulting composition to be formed into an appropriate storage form, facilitating disintegration in a liquid, or the like. Excipients can include, by way of illustration and not by limitation, diluents, disintegrants, binding agents, adhesives, wetting agents, lubricants, glidants, dyes, and any other substance other than the active ingredient conventionally used in the preparation of a liquid or solid formulation.

As used herein, “admixed” means that at least two components of the composition can be partially or fully mixed, dispersed, suspended, dissolved, or emulsified in one another. In some cases, at least a portion of the drug may be admixed in at least one carrier substance.

An initial overview of embodiments is provided below, and specific embodiments are then described in further detail. This initial summary is intended to aid readers in understanding the disclosure more quickly and is not intended to identify key or essential technological features, nor is it intended to limit the scope of the claimed subject matter.

### DETAILED DESCRIPTION

Many liquid formulations can include, among other things, an active agent dispersed in a liquid carrier such as, for example, a buffer solution. Liquid formulations, however, suffer from several disadvantages. For example, liquid formulations can be inconvenient, in part due to their increased liquid volume, to store and transport. Additionally, many active agents can react with a liquid carrier or other excipients more readily in liquid form, thus potentially reducing the potency/efficacy of the liquid formulation.

One solution to such problems can include maintaining the active agent and potentially other excipients as dry ingredients (i.e., the “dry form”) for transport and storage. When a liquid formulation is needed, the dry ingredients can be mixed with a liquid carrier. In this manner, the bulk volume of the transported portion of the formulation is minimized while maintaining the potency of the active agent. It can be convenient to include at least certain of the liquid formulation ingredients in the dry form. For example, buffering agents included in the dry form can greatly facilitate the process of mixing dry form ingredients with the liquid carrier prior to use, particularly if the dry form is in a premixed container that can be merely added to an appropriate volume of the liquid carrier. Many active agents, however, can react with other ingredients, such as buffering agents, that are present in the dry form.

The present disclosure provides formulations and systems having a storage form and usage form. As used herein, “usage form” refers to a liquid formulation prepared for use, such as, for example, ready to administer to a subject. The storage form, on the other hand, refers to a formulation that is not ready for use. Such storage forms include at least two ingredients where at least one of the formulation ingredients is concentrated to a powder, compressed powder, solid, or the like. Storage forms can additionally include all formulation ingredients concentrated to a powder, compressed powder, solid, or the like. In some examples, storage forms can include two or more formulation ingredients that react with one another, either immediately or over a period of time.

The present disclosure also provides methods of treating various disorders associated with mitochondrial dysfunction, including but not limited to metabolic disorders, neurodegenerative diseases, chronic inflammatory diseases, diseases of aging, photoaging, hyperproliferative diseases, including psoriasis and cancer, and the like.

The terms “reaction” and “react” include any form of chemical change that occurs to a formulation ingredient as a result of contact with another formulation ingredient, including reactions that activate one or more molecules or ingredients (e.g., the change of a precursor to an active agent into the active agent), reactions that degrade at least one ingredient, or the like.

In some cases, the usage form is a liquid formulation including an active agent and a reactive ingredient that, when in the storage form, is reactive with the active agent. The storage form is a concentrated formulation of the active agent and the reactive ingredient that has been formulated to reduce or eliminate the reactivity between the active agent and the reactive ingredient. Such a storage form is thus capable of long-term storage until, when ready for use, the usage form (i.e. the liquid formulation) is generated by the mixing the concentrated formulation of the storage form with an appropriate liquid carrier (e.g. water).

The storage forms of the present disclosure can vary depending on the natures of the intended liquid formulation, the active agent, excipients, any reactive ingredients, or the like. Such storage forms can be anhydrous or dry.

Various techniques can be used to reduce or eliminate the reactivity between the active agent and the reactive ingredient in the storage form. In one nonlimiting example of a storage form of the present disclosure, the active ingredient is admixed with the reactive ingredient and stored in a manner that reduces reaction between the active agent and the reactive ingredient. In such an example, the active agent and the reactive ingredient can be admixed together in the storage form, which can include storage at certain temperatures, package and storage in an oxygen-free and/or moisture-free environment, and the like. In some cases, the storage form can merely be used within a time window in which a degree of reaction between the two is acceptable.

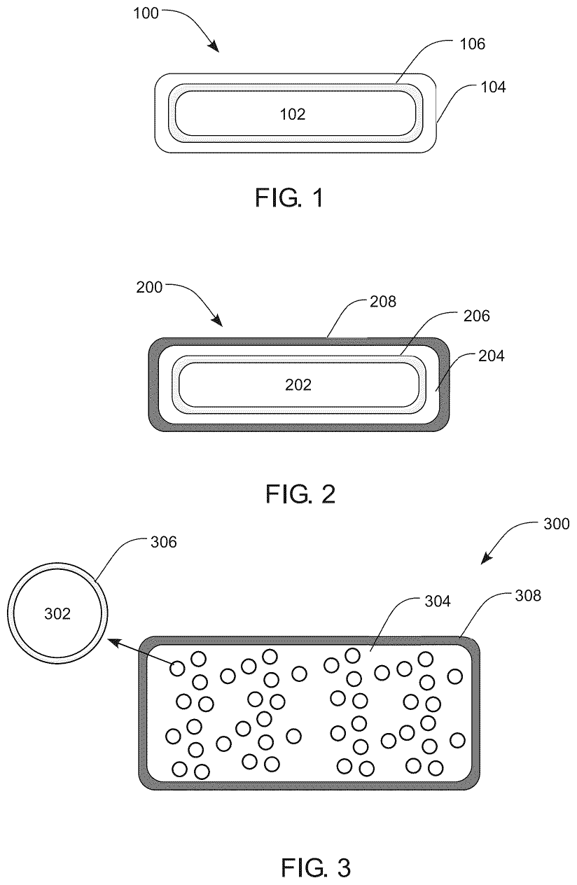

In another example, a first ingredient and a second reactive ingredient that react with one another can be separated from one another in a storage form by a reactivity isolation barrier. In FIG. 1, for example, a tablet 100 is shown including a first ingredient 102 and a second ingredient 104, where the first and second ingredients react with one another. To avoid such reactivity in the storage form, the first ingredient 102 and second ingredient 104 can be reactively separated from one another by a reactivity isolation barrier 106. The reactivity isolation barrier 106 thus reactively isolates the first reactive ingredient 102 from the second reactive ingredient 104. In some examples, the reactivity isolation barrier can be comprised of a disintegrable material that disintegrates when contacting a liquid carrier. When the tablet 100 is introduced into a liquid, the second reactive ingredient 104 disintegrates and/or dissolves into the liquid, followed by the disintegration and/or dissolution of the reactivity isolation barrier 106, which releases the active agent 102 into the liquid to form the liquid formulation of the usage form. In some examples, the first ingredient 102 can be in a gel or concentrated liquid form. In another example, a tablet is contemplated having the second ingredient at the center of the tablet surrounded by the reactivity isolation barrier, which is in turn surrounded by the first ingredient.

In a more specific example of FIG. 1, a tablet 100 can include an active agent 102, a reactive ingredient 104, and a reactivity isolation barrier 106 disposed between the active agent 102 and the reactive ingredient 104. The reactivity isolation barrier 106 thus reactively isolates the active agent 102 from the reactive ingredient 104. When the tablet 100 is introduced into a liquid, the reactive ingredient 104 disintegrates and/or dissolves into the liquid, followed by the disintegration and/or dissolution of the reactivity isolation barrier 106 and then the active agent 102 to form the liquid formulation of the usage form. In some examples, the active agent 102 can be in a gel or concentrated liquid form. In another example, a tablet is contemplated having the reactive ingredient at the center of the tablet surrounded by the reactivity isolation barrier, which is in turn surrounded by the active agent.

The reactivity isolation barrier can be any material layer that is pharmaceutically acceptable and that can reactively isolate the active agent from the reactive agent. In some examples, the reactivity isolation barrier can be or include a molecule, compound, or the like, that is an intended component of the resulting liquid formulation. For example, the disintegrable material can be or can include an excipient that does not react or does not substantially react with the active agent in the storage form. In other examples, the reactivity isolation barrier can be a molecule, compound, or the like, that provides an unintended beneficial effect to the liquid formulation. In yet other examples, the reactivity isolation barrier can be a molecule, compound, or the like, that has no effect or no substantial effect on the liquid formulation.

As another example, FIG. 2. shows a tablet 200 including an active agent 202, a reactive ingredient 204, and an inner reactivity isolation barrier 206 disposed between the active agent 202 and the reactive ingredient 204. The inner reactivity isolation barrier 206 thus reactively isolates the active agent 202 from the reactive ingredient 204. The tablet additionally includes an outer reactivity isolation barrier 208 to, at least in one aspect, provide a protective coating around the reactive ingredient 204.

The outer reactivity isolation barrier 208 can be the same material as the inner reactivity isolation barrier 206 or a different material from the inner reactivity isolation barrier 206. It is additionally contemplated that the outer reactivity isolation barrier 208 can include the same material as the inner reactivity isolation barrier 206 along with a different material that is not present in the inner reactivity isolation barrier 206. In other examples, the inner reactivity isolation barrier 206 can include the same material as the outer reactivity isolation barrier 208 along with a different material that is not present in the outer reactivity isolation barrier 208.

When the tablet 200 is introduced into a liquid, the outer reactivity isolation barrier 208 disintegrates and/or dissolves into the liquid, thus exposing the reactive ingredient 204. Once exposed to the liquid, the reactive ingredient 204 disintegrates and/or dissolves into the liquid, followed by the disintegration and/or dissolution of the inner reactivity isolation barrier 206 and the active agent 202 to form the liquid formulation of the usage form. In some examples, the active agent 202 can be in a gel or concentrated liquid form. In another example, a tablet is contemplated having a reactive ingredient at the center of the tablet surrounded by a reactivity isolation barrier, which is in turn surrounded by the active agent and the outer reactivity isolation barrier. It is additionally noted that the active agent and the reactive ingredient can be present in any of the above-mentioned regions/barriers, provided the active agent is reactively isolated from the reactive ingredient.

As yet another example, FIG. 3. shows a solid formulation 300 including a plurality of active agent particles 302 dispersed in a reactive ingredient 304, where each active agent particle 302 is surrounded by an inner reactivity isolation barrier 306 to reactively isolate the active agent in each active agent particle 302 from the reactive ingredient 304. In some examples, the solid formulation 300 can additionally include an outer reactivity isolation barrier 308 to, in at least in one aspect, provide a protective coating around the reactive ingredient 304. The outer reactivity isolation barrier 308 can be the same material as the inner reactivity isolation barrier 306 or a different material from the inner reactivity isolation barrier 306. It is additionally contemplated that the outer reactivity isolation barrier 308 can include the same material as the inner reactivity isolation barrier 306 along with a different material that is not present in the inner reactivity isolation barrier 306. In other examples, the inner reactivity isolation barrier 306 can include the same material as the outer reactivity isolation barrier 308 along with a different material that is not present in the outer reactivity isolation barrier 308.

In one example, the reactive ingredient 304 can be in a solid tablet form. In another example, the reactive ingredient 304 can be in a powder form. In one specific example of such a powder form, the outer reactivity isolation barrier 308 can contain the reactive ingredient 304 in a discrete dosage form. When the formulation is introduced into a liquid, the outer reactivity isolation barrier 308 dissolves or otherwise breaks down into the liquid, thus exposing the powdered reactive ingredient 304. Once exposed to the liquid, the reactive ingredient 304 and the inner reactivity isolation barrier 306 surrounding each active agent particle 302 dissolves or otherwise breaks down into the liquid, thus exposing the active agent, which in turn dissolves or breaks down to form the liquid formulation of the usage form. Such dosage form can generate the liquid formulation much more rapidly than a tablet form due to the liquid diffusing more rapidly through the powdered reactive ingredient 304 and the greatly increased surface area of the reactivity isolation barrier and the active agent portions of the storage form. In examples where an outer reactivity isolation barrier is not present, the reactive ingredient 304 containing the plurality of active agent particles 302 can be utilized as a powder.

In another example, a solid formulation is contemplated having a plurality of reactive ingredient particles dispersed in an active agent where each reactive ingredient particle is surrounded by an inner reactivity isolation barrier to reactively isolate the reactive ingredient particles from the active agent. It is additionally noted that the active agent and the reactive ingredient can be present in any of the above mentioned regions/barriers, provided the active agent is reactively isolated from the reactive ingredient.

In some cases, the present storage form can include a pharmaceutically acceptable diluent, for example, as a filler to increase weight, improve content uniformity, or the like. Suitable diluents include, either individually or in combination and without limitation, lactose; lactose, anhydrous; lactose monohydrate; starch; directly compressible starch; hydrolyzed starch; partially pregelatinized starch; sodium starch glycolate; mannitol; sorbitol; xylitol; dextrose monohydrate; dibasic calcium phosphate dihydrate; sucrose-based diluents; confectioner's sugar; monobasic calcium sulfate monohydrate; calcium sulfate dihydrate NF; calcium lactate trihydrate granular; dextrates; dextrose; inositol; hydrolyzed cereal solids; amylase; powdered cellulose; calcium carbonate; glycine; bentonite; polyvinylpyrrolidone; clays;

celluloses; purified cellulose; methylcellulose; sodium carboxymethylcellulose, carboxymethylcellulose; microcrystalline cellulose; alginates; pregelatinized corn starches; crospovidone; gums; agar; guar; locust bean; Karaya; pectin; tragacanth; and the like. The use of extra-granular microcrystalline cellulose (i.e., microcrystalline cellulose added to a wet granulated composition after the drying step), for example, can be used to improve hardness and/or disintegration time. Many diluents provide storage forms having suitable disintegration rates, stability, pre-compression flowability, and/or drying properties. Diluents can also provide high density substrate that aid densification during granulation (where wet granulation is employed) and, therefore, improve blend flow properties.

In some cases, the present storage form can include a pharmaceutically acceptable binder or adhesive. Such binding agents and adhesives can provide sufficient cohesion to a powder being tableted to allow for improved processing operations such as sizing, lubrication, compression and packaging, but still allow the tablet to disintegrate. Suitable binding agents and adhesives include, either individually or in combination and without limitation, acacia; tragacanth; sucrose; gelatin; glucose; starch; cellulose materials such as, but not limited to, cellulose, microcrystalline cellulose, cellulose ethers, hydroxypropyl cellulose, methylcellulose, sodium carboxymethylcellulose; ethyl-cellulose; alginic acid and salts of alginic acid; magnesium aluminum silicate; polyethylene glycol; guar gum; polysaccharide acids; bentonites; polyvinylpyrrolidone; polymethacrylates; hydroxypropylmethyl-cellulose; hydroxypropylcellulose; pregelatinized starch; saccharides and appropriate derivatives; disaccharides; sucrose; lactose; polysaccharides and appropriate derivatives; sugar alcohols such as, without limitation, xylitol, sorbitol or mannitol; protein; gelatin, and the like.

Solution binders can be dissolved in a solvent used in wet granulation processes. Examples of solution binders can include gelatin, cellulose, cellulose derivatives, polyvinylpyrrolidone, starch, sucrose, polyethylene glycol, and the like. Dry binders can be added to a powder blend, either after a wet granulation step or as part of a direct powder compression formulation. Examples of dry binders can include cellulose, methyl cellulose, polyvinylpyrrolidone, polyethylene glycol, and the like.

Binder and/or adhesives can be present in any amount sufficient to achieve a desired result. In one example, a binder and/or adhesive can constitute from about 0.5% to about 25% of the total weight of a storage form. In another example, a binder and/or adhesive can constitute from about 0.75% to about 15% of the total weight of a storage form. In yet another example, a binder and/or adhesive can constitute from about 1% to about 10% of the total weight of a storage form. As one specific example, polyvinylpyrrolidone can be used to impart cohesive properties to a powder blend of an active agent and other excipients for granulation. Polyvinylpyrrolidone can be present in any amount sufficient to achieve a desired result. In one example, polyvinylpyrrolidone can constitute from about 0.5% to about 10% of the total weight of a storage form. In another example, polyvinylpyrrolidone can constitute from about 0.5% to about 7% of the total weight of a storage form. In yet another example, polyvinylpyrrolidone can constitute from about 0.5% to about 5% of the total weight of the composition.

In some cases, the present storage form can include a pharmaceutically acceptable disintegrant to facilitate disintegration of the storage form into the liquid formulation when added to an appropriate solvent. Suitable disintegrants include, either individually or in combination and without limitation, starch such as cornstarch, rice starch, sodium starch glycolate, and the like; cross-linked N-vinyl Base-2-Pyrrolidone (CLPVP); alginic acid or alginate; microcrystalline cellulose; hydroxypropyl cellulose and other celluloses; cross-linked sodium carboxymethyl cellulose, such as croscarmellose sodium; polyvinyl polypyrrolidone; crospovidone; polacrilin potassium, and the like. In one example, the disintegrant can be a gas-producing disintegrant, such as and without limitation, sodium bicarbonate; potassium bicarbonate; sodium acid carbonate; citric acid; tartaric acid, and the like.

Disintegrants can be present in any amount sufficient to achieve a desired result and can be added at any suitable step during the preparation of the storage form, in some cases prior to granulation or during a lubrication step prior to compression. In one example, a disintegrant can constitute from about 0.2% to about 30% of the total weight of the composition. In another example, a disintegrant can constitute from about 0.2% to about 10% of the total weight of the composition. In yet another example, a disintegrant can constitute from about 0.2% to about 5% of the total weight of the composition. In some cases, a disintegrant can be admixed with the active agent prior to granulation. In some cases, the disintegrant can be divided into two portions: one part is added to a powdered formula prior to granulation and the remainder is admixed with a lubricant and added prior to compression. In this manner, the portion added to the lubricant rapidly breaks down the tablet granules and disintegrant mixed with the active ingredients disintegrates the granules into smaller particles.

In one example, the storage form of the formulation can be an effervescent formulation, such as an effervescent tablet, an effervescent powder, or the like. An effervescent tablet can be coated or uncoated, depending on the tablet design. Any effervescent material that is compatible with the formulation and that can react in the presence of water to release a gas that facilitates disintegration of the storage form can be suitable for use. In one example, the effervescent material can react in the presence of water to release carbon dioxide. In some nonlimiting examples, effervescent materials can include acidic substances, such as citric acid, tartaric acid, malic acid, fumaric acid, adipic acid, or any other suitable acid, including anhydrides and salts thereof. In other nonlimiting examples, effervescent materials can include citric acid, tartaric acid, or malic acid, including anhydrides and salts thereof. In further examples, effervescent materials can include carbonates, bicarbonates, or the like. More specific examples can include potassium carbonate, sodium carbonate, sodium bicarbonate, potassium bicarbonate, arginine carbonate, and the like.

In some cases, the present storage form can include a pharmaceutically acceptable tablet coating to protect tablet ingredients from deterioration by moisture in the air. Nonlimiting examples of such coatings can include a cellulose ether hydroxypropyl methylcellulose (HPMC) film, various synthetic polymers, shellac, corn protein zein, other polysaccharides, and the like.

In some examples, an active agent of the present disclosure can inhibit energy within certain cells to treat various conditions. In one specific example, the active agent can be a cellular energy inhibitor according to Formula I:

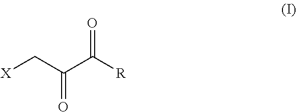

Various specific molecules are contemplated, wherein, for example, X can be, without limitation, a nitro, an imidazole, a halide, sulfonate, a carboxylate, an alkoxide, amine oxide, or the like. Additionally, R can be, without limitation, OR′, N(R″)₂, C(O)R″′, C1-C6 alkyl, C6-C12 aryl, C1-C6 heteroalkyl, a C6-C12 heteroaryl, H, an alkali metal or the like, where R′ represents H, alkali metal, C1-C6 alkyl, C6-C12 aryl or C(O)R″′, R″ represents H, C1-C6 alkyl, or C6-C12 aryl, and R′″ represents H, C1-C20 alkyl or C6-C12 aryl.

In one example, R of formula (I) can be OH and X of formula (I) can be a nitro, an imidazole, a halide, a sulfonate, a carboxylate, an alkoxide, an amine oxide, or the like. Additionally, X can be a halide, such as, for example, fluoride, bromide, chloride, iodide, or the like. In one example, X can be a sulfonate, such as, for example, a triflate, a mesylate, a tosylate, or the like. In another example, X can be amine oxide. In still another example, the amine oxide can be dimethylamine oxide.

In another example, the cellular energy inhibitor can be a 3-halopyruvate, such as, for example, 3-fluoropyruvate, 3-chloropyruvate, 3-bromopyruvate, 3-iodopyruvate, or a combination thereof. A general structure showing a halide in the 3-position is shown in formula II.

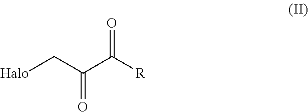

In a further nonlimiting example, the cellular energy inhibitor can have bromine in the 3-position, as shown in formula III.

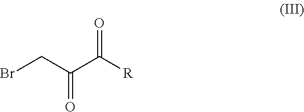

In one further nonlimiting example, the cellular energy inhibitor can be 3-bromopyruvic acid (3-BP), as shown in formula IV.

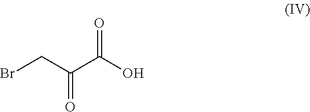

In another nonlimiting example, the cellular energy inhibitor can be 3-bromopyrate, as shown in formula V.

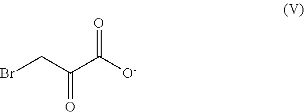

In some examples the cellular energy inhibitor can be formulated in a composition with at least one sugar, which can stabilize the cellular energy inhibitor by substantially preventing the inhibitor from hydrolyzing. In some examples, a composition can include 3-BP and at least one sugar, at least two sugars, at least three sugars, and the like. In one example, a sugar can include a monosaccharide, a disaccharide, an oligosaccharide, or a combination thereof. Nonlimiting examples of monosaccharides can include glucose, fructose, galactose, etc. Nonlimiting examples of disaccharides can include sucrose, lactose, maltose, etc. It is noted that, for the purposes of the present disclosure, the term “sugar” can also include oligosaccharides, polysaccharides, polyols, polyalcohols, and similar molecules that function to stabilize 3-BP.

A sugar can include a 3-carbon sugar, a 4-carbon sugar, a 5-carbon sugar, a 6-carbon sugar, a 7-carbon sugar, and the like, including combinations thereof. In one aspect, the sugar can be a 3-carbon sugar, a 4-carbon sugar, a 5-carbon sugar, a 6-carbon sugar, a 7-carbon sugar, and the like, including combinations thereof, provided the sugar is not involved in energy metabolism to the extent that it generates energy (i.e., a nonmetabolizable sugar).

In one example, the sugar can be gluconic acid. In another example, the sugar can be glucuronic acid. At least one of the sugars can be a five-carbon sugar. In one example, at least two of the sugars can be five-carbon sugars. The five-carbon sugars can be independently selected from mannitol, erythritol, isomalt, lactitol, maltitol, sorbitol, xylitol, dulcitol, ribitol, inositol, myo-inositol or the like, including combinations thereof. In one example, at least one of the sugars can be glycerol. In another example, the sugars can be glycerol, inositol, and sorbitol. Other nonlimiting example of sugars can include ethylene glycol, threitol, arabitol, galactitol, fucitol, iditol, volemitol, maltotriitol, maltotetraitol, and polyglycitol, including combinations thereof. In one example, the sugars can include glycerol, inositol, myo-inositol, sorbitol, mannitol or any combination thereof. In another example, the sugars can include glycerol, inositol, sorbitol, or any combination thereof. In yet another example, the inositol can be myo-inositol. In other examples, the sugar can be a polyalcohol. In another example, the sugars can include a heptasaccharide such as, without limitation, a cyclodextrin, such as beta-cyclodextrin.

The sugars described herein can be any isomeric form. In one example, the compositions described herein can include the less biologically active form of the sugar as compared to its isomer. In one case, the less biologically active sugar can be the L-enantiomer sugar. However, if the D-enantiomer sugar is found to be less biologically active as compared to its L form, then the D form can be used. In one example, such sugars can function as a glycolytic inhibitor.

In one example, a composition can include one or more sugars in a range from about 0.5 wt % to about 50.0 wt % or from about 1.0 wt % to about 25.5 wt %. In yet another example, a composition can include one or more sugars in a range from about 0.2 wt % to about 75.0 wt % or from about 0.5 wt % to about 50.0 wt %. In a further example, a composition can include one or more sugars in a range from about 0.1 wt % to about 25.0 wt %, from about 0.2 wt % to about 10.0 wt %.

In some examples, the composition can include glycerol in a range from about 0.1 wt % to about 5.0 wt % or from about 0.1 wt % to about 3.0 wt %. In other examples, the composition can include inositol in a range from about 0.1 wt % to about 10 wt %, from about 0.1 wt % to about 6 wt %. In further examples, the composition can include sorbitol in a range from about 0.1 wt % to about 40.0 wt % or from about 0.1 wt % to about 30 wt %. In yet further examples, the composition can include mannitol in a range from about 0.1 wt % to about 30 wt % or from about 0.1 wt % to about 10 wt %. Additionally, each of the sugars may be added in a volume up to a maximum solubility of the sugar in the formulation or composition. It is additionally noted that the above wt % s of ingredients are without water or other liquid carrier.

In some examples, a 3-BP composition can include a biological buffer that is present in an amount sufficient to at least partially deacidify the cellular energy inhibitor and neutralize metabolic by-products of the cellular energy inhibitor. Nonlimiting examples of biological buffers can include a citrate buffer, a phosphate buffer, an acetate buffer, or the like, including combinations thereof. In one specific example, the biological buffer can be a citrate buffer, such as, without limitation, sodium citrate. In another specific example, the biological buffer can be a phosphate buffer, such as, without limitation, sodium phosphate. In one specific example, the biological buffer can be an acetate buffer, such as, without limitation, sodium acetate. In yet other examples, the biological buffer can include at least two biological buffers, such as, without limitation, a citrate buffer and an acetate buffer, a citrate buffer and a phosphate buffer, an acetate buffer and a phosphate buffer, or a citrate buffer, a phosphate buffer, and an acetate buffer.

In some examples, the composition can include the biological buffer in a range of from about 0.1 wt % to about 15 wt % or from about 2.0 wt % to about 8.0 wt %. Additionally, the biological buffer can maintain a physiological pH of 4.0 to 8.5. In one embodiment, the biological buffer can maintain a physiological pH of 5.5 to 8.0. In another embodiment, the biological buffer can maintain a physiological pH of 6.8 to 7.8. In still another embodiment, the biological buffer can maintain a physiological pH of 7.3 to 7.6. It is additionally noted that the above wt % s of ingredients are without water or other liquid carrier.

In one example, 3-BP can be admixed with the biological buffer without a reactivity isolation barrier between the two. Additionally, other ingredients, such as the sugar(s), for example, can be admixed with the 3-BP and the biological buffer. In some examples, the admixed solid formulation can include an outer protective coating. In other examples, the admixed solid formulation lacks an outer protective coating.

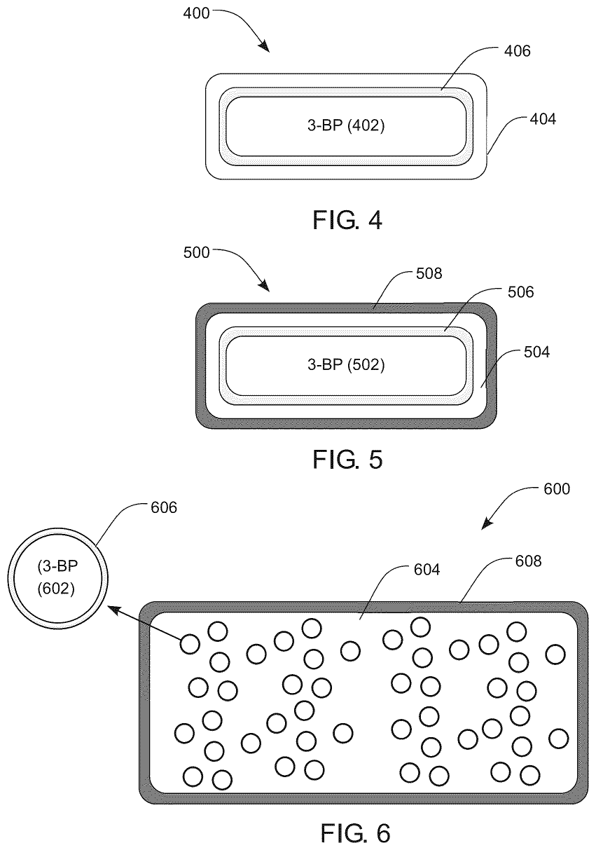

In one example, 3-BP can be included in a storage form with a biological buffer that are reactively isolated from one another by a reactivity isolation barrier. FIG. 4, for example, shows a tablet 400 including 3-BP 402, a biological buffer 404, and a reactivity isolation barrier 406 disposed between the 3-BP 402 and the biological buffer 404. The reactivity isolation barrier 406 thus reactively isolates the 3-BP 402 from the biological buffer 404. When the tablet 400 is introduced into a liquid, the biological buffer 404 dissolves or otherwise disintegrates into the liquid, followed by the dissolution or disintegration of the reactivity isolation barrier 406 and then the 3-BP 402 to form the liquid formulation of the usage form. In another example, a tablet is contemplated having a biological buffer at the center of the tablet surrounded by a reactivity isolation barrier, which is in turn surrounded by 3-BP.

The reactivity isolation barrier can be any material layer that is pharmaceutically acceptable and that can reactively isolate 3-BP from the biological buffer. In some examples, the reactivity isolation barrier can be a molecule, compound, or the like, that is an intended component of the resulting liquid formulation. For example, the disintegrable material can be or can include an excipient that does not react or does not substantially react with 3BP. In other examples, the reactivity isolation barrier can be a molecule, compound, or the like, that provides an unintended beneficial effect to the liquid formulation. In yet other examples, the reactivity isolation barrier can be a molecule, compound, or the like, that has no effect or no substantial effect on the liquid formulation.

In one specific example, the reactivity isolation barrier includes at least one sugar, at least two sugars, at least three sugars, etc., of the desired 3-BP liquid formulation. In another example, the sugar(s) can be admixed with the 3-BP. In yet another example, the sugar(s) can be admixed with the biological buffer. In yet another example, the sugars can be added to the liquid formulation following disintegration of the storage form or to the liquid prior to disintegration of the storage form.

As another example, FIG. 5. shows a tablet 500 including 3-BP 502, a biological buffer 504, and an inner reactivity isolation barrier 506 disposed between the 3-BP 502 and the biological buffer 504. The inner reactivity isolation barrier 506 thus reactively isolates the 3-BP 502 from the biological buffer 504. The tablet additionally includes an outer reactivity isolation barrier 508 to, at least in one aspect, provide a protective coating around the biological buffer 504. The outer reactivity isolation barrier 508 can be the same material as the inner reactivity isolation barrier 506 or a different material as the inner reactivity isolation barrier 506. It is additionally contemplated that the outer reactivity isolation barrier 508 can include the same material as the inner reactivity isolation barrier 506 along with a different material that is not present in the inner reactivity isolation barrier 506. In other examples, the inner reactivity isolation barrier 506 can include the same material as the outer reactivity isolation barrier 508 along with a different material that is not present in the outer reactivity isolation barrier 508. When the tablet 500 is introduced into a liquid, the outer reactivity isolation barrier 508 dissolves or otherwise breaks down into the liquid, thus exposing the biological buffer 504. Once exposed to the liquid, the biological buffer 504 dissolves or otherwise breaks down into the liquid, followed by the dissolutions or breaking down of the inner reactivity isolation barrier 506 and the 3-BP 502 to form the liquid formulation of the usage form. In some examples, the 3-BP 502 can be in a gel or concentrated liquid form. In another example, a tablet is contemplated having a biological buffer at the center of the tablet surrounded by a reactivity isolation barrier and further surrounded by the 3-BP which is in turn surrounded by an outer reactivity isolation barrier.

In one specific example, the inner reactivity isolation barrier, the outer reactivity isolation barrier, or both, can include at least one sugar, at least two sugars, at least three sugars, etc., of the desired 3-BP liquid formulation. In another example, the sugar(s) can be admixed with the 3-BP. In yet another example, the sugar(s) can be admixed with the biological buffer. It is additionally contemplated that the inner reactivity isolation barrier and/or the outer reactivity isolation barrier can include one or more sugars. In some examples, the one or more sugars in each reactivity isolation barrier can be the same, different, or various mixtures thereof. In yet another example the sugars can be added to the liquid formulation following disintegration of the storage form or to the liquid prior to disintegration of the storage form.

As yet another example, FIG. 6. shows a solid formulation 600 including a plurality of 3-BP particles 602 dispersed in a biological buffer 604, where each of the 3-BP particles 602 is surrounded by an inner reactivity isolation barrier 606 to reactively isolate the active agent in each 3-BP particles 602 from the biological buffer 604. In some examples, the solid formulation 600 can additionally include an outer reactivity isolation barrier 608 to, in at least in one aspect, provide a protective coating around the biological buffer 604.

The outer reactivity isolation barrier 608 can be the same material as the inner reactivity isolation barrier 606 or a different material from the inner reactivity isolation barrier 606. It is additionally contemplated that the outer reactivity isolation barrier 608 can include the same material as the inner reactivity isolation barrier 606 along with a different material that is not present in the inner reactivity isolation barrier 606. In other examples, the inner reactivity isolation barrier 606 can include the same material as the outer reactivity isolation barrier 608 along with a different material that is not present in the outer reactivity isolation barrier 608.

In one example, the biological buffer 604 can be in a solid tablet form. In another example, the biological buffer 604 can be in a powder form. In one specific example of such a powder form, the outer reactivity isolation barrier 608 can contain the biological buffer 604 in a discrete dosage form similar to a capsule. When the capsule is introduced into a liquid, the outer reactivity isolation barrier 608 dissolves or otherwise breaks down into the liquid, thus exposing the powdered biological buffer 604. Once exposed to the liquid, the biological buffer 604 and the reactivity isolation barrier 606 surrounding each 3-BP particles 602 dissolves or otherwise breaks down into the liquid, thus exposing the 3-BP, which in turn dissolves or breaks down to form the liquid formulation of the usage form. Such dosage form can generate the liquid formulation much more rapidly than a tablet form due to the liquid diffusing more rapidly through the powdered reactive ingredient 604 and the greatly increased surface area of the reactivity isolation barrier and the active agent portions of the storage form. In examples where an outer reactivity isolation barrier is not present, the reactive ingredient 604 containing the plurality of 3-BP particles 602 can be utilized as a powder. In yet another example, the powder can be a compressed powder or solid.

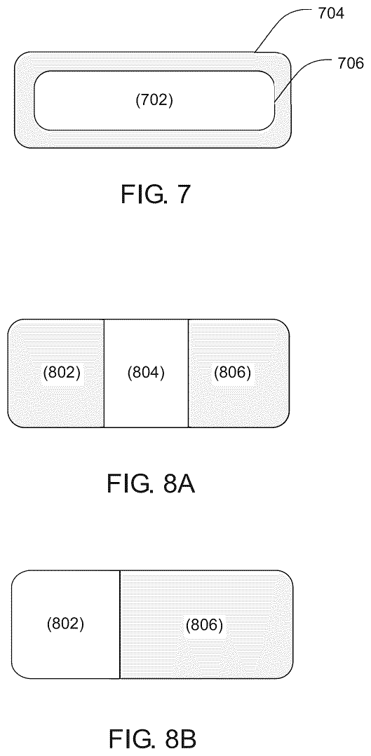

In addition to providing a reactivity isolation barrier between regions, ingredients that react with one another in the storage form can be in direct contact therebetween along a common border. Reactivity between an active agent and a reactive ingredient is thus minimized by limiting reactivity to a proportionally small region of the storage form, i.e., along the common boarder. In one example shown in FIG. 7, an active agent 702 is surrounded by a reactive ingredient 740. While reaction can occur between the active agent 702 and the reactive agent 704, such is limited to the common border 706. In addition to the described arrangement of ingredients, it is additionally contemplated that the reactive ingredient can be surrounded by the active agent.

FIGS. 8A & 8B show examples having distinct regions of ingredients positioned adjacent one another. FIG. 8A, for example, shows a storage form having three ingredients, 802, 804, and 806 arranged side-by-side. Any reactivity that occurs between these ingredients is thus limited to the common boarders therebetween. FIG. 8B shows a similar storage form arrangement having two ingredients, 802 and 806.

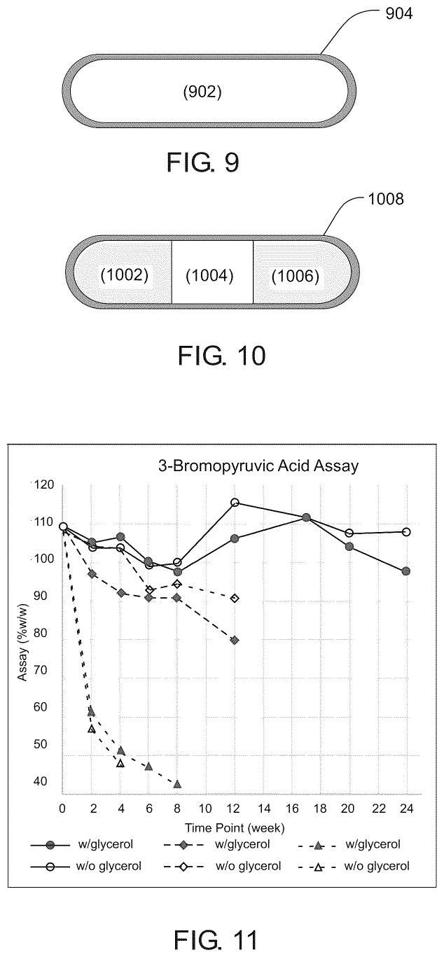

In another example, a storage form can be contained within a capsule, for example and without limitation, either in a solid compacted form or a powdered form. FIG. 9 shows a capsule 904 containing an admixture 902 of a cellular energy inhibitor or a cellular energy inhibitor precursor along with at least one other excipient in a powdered form, a compacted form, or the like. In some cases, a capsule storage form can be directly induced into a liquid carrier to form the usage form. In other cases, the capsule can be opened and it's contents can be poured into the liquid carrier to form the usage form. Capsules can be FIG. 10 shows an example of a capsule 1008 containing a storage form having three partitioned ingredients, 1002, 1004, and 1006.

Various components of a composition in a storage form can also react when exposed to oxygen and other reactive elements/molecules in the air, including moisture in some cases. As such, in one technique reactivity can be minimized or prevented by vacuum packing the storage form. In another technique, reactivity can be minimized or prevented by packaging the storage form in an inert gas. While the solid form can be packaged in any package form, such as bottles, tubes, pouches, and the like, one convenient package form is a blister pack. A blister pack allows the solid form to be each discrete storage form dose to be used without exposing the remaining doses in a blister pack to the air/moisture in the local environment.

In one specific example, the reactivity isolation barrier can include at least one sugar, at least two sugars, at least three sugars, etc., of the desired 3-BP liquid formulation. In another example, the sugar(s) can be admixed with the 3-BP. In yet another example, the sugar(s) can be admixed with the biological buffer. It is additionally contemplated that the reactivity isolation barrier and/or the outer reactivity isolation barrier can include one or more sugars that are either the same, different, or various mixtures of sugars in a single barrier or in each barrier. In yet another example, the sugars can be added to the liquid formulation following disintegration of the storage form or to the liquid prior to disintegration of the storage form.

In one specific example, a storage form can include a powder admixture of at least 3-BP, at least one buffer, at least one sugar, and excipients. Such a mixed powder storage form has an extended stability profile, particularly compared to a liquid usage form having similar ingredients. 3-BP in such a mixed powder formulation shows only slight degradation, for example, after 24 weeks or more at −20° C.

As one example, stability of a 3-BP mixed powder formulation is increased over time as a function of storage temperature, particularly when stored in an inner packaging material and an outer packaging material. For example, an inner packaging material can include a polyethylene pouch and the outer primary packaging material can include an aluminum pouch. Table 1 shows one example of a 3-BP mixed powder formulation at different storage temperatures over time, where the 3-BP mixed powder formulation includes 3-BP, sodium citrate, sodium phosphate, myo-inositol, sorbitol, glycerol, microcrystalline cellulose, colloidal silicon dioxide, and steric acid. Table 1 shows one example of a 3-BP mixed powder formulation at different storage temperatures over time, where the 3-BP mixed powder formulation includes 3-BP, sodium citrate, sodium phosphate, myo-inositol, sorbitol, microcrystalline cellulose, colloidal silicon dioxide, and steric acid. Testing assays for appearance, total 3-BP, and total impurity are indicators of the degradation of 3-BP in the 3-BP mixed powder formulations over time and at different temperatures, from room temperature to −20° C.

TABLE 1
Stability of 3-BP Mixed Powder Formulation with Glycerol

| | | Time point | | | | | | | |
|---|---|---|---|---|---|---|---|---|---|
| Storage | Test | 0 | 2-week | 4-week | 6-week | 8-week | 12-week | 20-week | 24-week |
| −20° C. | Appearance | White to | White to | White to | White to | White to | White to | White to | White to |
| | | off-white | off-white | off-white | off-white | off-white | off-white | off-white | off-white |
| | | powder | powder | powder | powder | powder | powder | powder | powder |
| | % w/w 3-BP | 109% | 105% | 106% | 100% | 97% | 106% | 104% | 97% |
| | Total impurity | 0.07% | 0.08% | 0.08% | 0.08% | 0.07% | 0.07% | 0.06% | 0.06% |
| 5° C. | Appearance | White to | White to | White to | White to | White to | White to | — | — |
| | | off-white | off-white | off-white | off-white | off-white | off-white | | |
| | | powder | powder | powder | powder | powder | powder | | |
| | % w/w 3-BP | 109% | 96% | 91% | 90% | 90% | 79% | — | — |
| | Total impurity | 0.07% | 0.12% | 0.08% | 0.09% | 0.12% | 0.13% | — | — |
| Room | Appearance | White to | White to | Brown | Brown | Brown | — | — | — |
| temp. | | off-white | off-white | powder | powder | powder | | | |
| | | powder | powder | | | | | | |
| | % w/w 3-BP | 109% | 60% | 51% | 46% | 42% | — | — | — |
| | Total impurity | 0.07% | 0.34% | 0.33% | 0.42% | 0.39% | — | — | — |

TABLE 2
Stability of 3-BP Mixed Powder Formulation without Glycerol

| | | Time point | | | | | | | |
|---|---|---|---|---|---|---|---|---|---|
| Storage | Test | 0 | 2-week | 4-week | 6-week | 8-week | 12-week | 20-week | 24-week |
| −20° C. | Appearance | White to | White to | White to | White to | White to | White to | White to | White to |
| | | off-white | off-white | off-white | off-white | off-white | off-white | off-white | off-white |
| | | powder | powder | powder | powder | powder | powder | powder | powder |
| | % w/w 3-BP | 109% | 103% | 103% | 99% | 99% | 115% | 107% | 107% |
| | Total impurity | 0.18% | 0.17% | 0.15% | 0.14% | 0.16% | 0.13% | 0.11% | 0.11% |
| 5° C. | Appearance | White to | White to | White to | White to | White to | White to | — | — |
| | | off-white | off-white | off-white | off-white | off-white | off-white | | |
| | | powder | powder | powder | powder | powder | powder | | |
| | % w/w 3-BP | 109% | 104% | 103% | 92% | 94% | 90% | — | — |
| | Total impurity | 0.18% | 0.17% | 0.17% | 0.17% | 0.17% | 0.20% | — | — |
| Room | Appearance | White to | Brown | Brown | — | — | — | — | — |
| temp. | | off-white | powder | powder | | | | | |
| | | powder | | | | | | | |
| | % w/w 3-BP | 109% | 56% | 47% | — | — | — | — | — |
| | Total impurity | 0.18% | 0.67% | 0.73% | — | — | — | — | — |

Turning to FIG. 11, both of the 3-BP formulations (with glycerol and without glycerol) show no significant degradation at −20° C. for 24 weeks, gradual degradation at 5° C. over 12 weeks, and significant degradation over 8 weeks. The assay labeled in FIG. 11 is the % w/w of 3-BP as determined by HPLC. In one example, at least 85% w/w of 3-BP remains in the formulation after 24 weeks at −20° C. In another example, at least 90% w/w of 3-BP remains in the formulation after 24 weeks at −20° C. In yet another example, at least 95% w/w of 3-BP remains in the formulation after 24 weeks at −20° C.

In some examples, a 3-BP composition can include a glycolysis inhibitor, one nonlimiting example of which can include 2-deoxglucose (2DOG). The 3-BP formulation can include the glycolysis inhibitor in any effective amount. In the various dosage forms described above in FIGS. 4-6, the glycolysis inhibitor can be admixed with the 3-BP or can be present in a separate layer or in any layer described above, provided the glycolysis inhibitor is isolated from any ingredient in the storage to which it reacts, such as, for example, the biological buffer. Provided it does not react with the biological buffer, the glycolysis inhibitor can be admixed therein.

In addition to the above components, the 3-BP compositions described herein can further comprise a halo monocarboxylate compound that is separate from the cellular energy inhibitor. In cases where the halo monocarboxylate compound can function to inhibit glycolysis and/or mitochondria function, the halo monocarboxylate can be considered a second cellular energy inhibitor. In one embodiment, the halo monocarboxylate compound can be a halo two-carbon monocarboxylate compound. The halo two-carbon monocarboxylate compound can be selected from, without limitation, 2-fluoroacetate, 2-chloroacetate, 2-bromoacetate, 2-iodoacetate, and the like, including combinations thereof. In one embodiment, the halo two-carbon monocarboxylate compound can be 2-bromoacetate. In one example, the composition can comprise the halo two-carbon monocarboxylate compound in a concentration from about 0.01 mM to about 5.0 mM. In another example, the composition can comprise a halo two-carbon monocarboxylate compound in a concentration from about 0.1 mM to about 0.5 mM.

Additionally, a halo monocarboxylate compound can be a halo three-carbon monocarboxylate compound. In one embodiment, the balo three-carbon monocarboxylate compound can be selected from, without limitation, 3-fluorolactate, 3-chlorolactate, 3-bromolactate, 3-iodolactate, and the like, including combinations thereof. In another example, the composition can include the halo three-carbon monocarboxylate compound in a concentration from about 0.5 mM to about 250 mM. In one embodiment, the composition can comprise the halo three-carbon monocarboxylate compound in a concentration from about 10 mM to about 50 mM. In the various dosage forms described above in FIGS. 4-7, the halo monocarboxylate compound can be admixed with the 3-BP or be present in a separate layer or in any layer described above, provided the halo monocarboxylate compound is reactively isolated in the storage form.

In some examples, the 3-BP compositions described herein can further comprise a mitochondrial inhibitor in addition to the cellular energy inhibitor. The mitochondrial inhibitor can be selected from, without limitation, oligomycin, efrapeptin, aurovertin, and the like, including combinations thereof. In another example, the composition can include the mitochondrial inhibitor in a concentration from about 0.001 mM to about 5.0 mM. In one example, the composition can include the mitochondrial inhibitor in a concentration from about 0.01 mM to about 0.5 mM. In the various dosage forms described above in FIGS. 4-7, the mitochondrial inhibitor can be admixed with the 3-BP or be present in a separate layer or in any layer described above, provided the mitochondrial inhibitor is reactively isolated in the storage form.

In some examples, the present 3-BP compositions can comprise antifungal agents, antibiotics, glycolysis inhibitors, inhibitors of mitochondria, sugars, and biological buffers, without limitation. Examples of such agents include, but are not limited to, amphotericin B, efrapeptin, doxorubicin, (2DOG), analogs of 2DOG, d-lactic acid, dichloroacetic acid (or salt form of dichloroacetate), oligomycin, analogs of oligomycin, glycerol, inositol, sorbitol, glycol, erythritol, threitol, arabitol, xylitol, ribitol, mannitol, dulcitol, iditol, isomalt, maltitol, lactitol, polyglycitol, sodium phosphate, sodium citrate, sodium acetate, sodium carbonate, sodium bicarbonate, sodium pyruvate, sodium lactate, oxaloacetate, isocitrate, aconitate, succinate, fumarate, malate, diluted saline solutions with varying concentrations of NaCl, and water. In addition to the sodium ion that accompanies these biological buffers, calcium and potassium cations can also accompany the biological buffers. Various active agents of the composition can include a cellular energy inhibitor, a glycolysis inhibitor, a mitochondria inhibitor, a halo monocarboxylate compound, an antifungal agent, an antibiotic agent, and the like. In the various dosage forms described above in FIGS. 4-7, any of the above ingredients can be admixed with the 3-BP or be present in a separate layer or in any layer described above, provided the added ingredient(s) is/are reactively isolated in the storage form.

In some examples, the 3-BP compositions described herein can further comprise a hexokinase inhibitor. In the various dosage forms described above in FIGS. 4-7, the hexokinase inhibitor can be admixed with the 3-BP or be present in a separate layer or in any layer described above, provided the hexokinase inhibitor is reactively isolated in the storage form.

As used herein, “hexokinase 1” or “hexokinase 1 isozyme” refers to any isoforms of hexokinase 1 and its naturally known variants, including those provided in SEQ ID NO: 1, SEQ ID NO: 2, SEQ ID NO: 3, and SEQ ID NO: 4, as follows:

```
(SEQ ID NO: 1)
    1 MIAAQLLAYY FTELKDDQVK KIDKYLYAMR LSDETLIDIM TRFRKEMKNG LSRDENPTAT
  61 VKMLPTFVRS IPDGSEKGDF IALDLGGSSP RILRVQVNHE KNQNVHMESE VYDTPENIVH
121 GSGSQLFDHV AECLGDFMEK RKIKDKKLPV GFTFSFPCQQ SKIDEAILIT WTKRFKASGV
181 EGADVVKLLN KAIKKRGDYD ANIVAVVNDT VGTMMTCGYD DQHCEVGLII GTGTNACYME
241 ELRHIDLVEG DEGRMCINTE WGAFGDDGSL EDIRTEEDRE IDRGSLNPGK QLFEKMVSGM
301 YLGELVRLIL VKMAKEGLLF EGRITPELLT RGKFNTSDVS AIEKNKEGLK NAKEILTRLG
361 VEPSDDDCVS VQHVCTIVSF RSANLVAATL GAILNRLRDN KGTPRLRTTV GVDGSLYKTH
421 PQYSRRFHKT LRRLVPDSDV RFLLSESGSG KGAAMVTAVA YRLAEQHRQI EETLAHFHLT
481 KDMLLEVKKR MRAEMELGLR KQTHNNAVVK MLPSFVRRTP DGTENGDFLA LDLGGTNERV
541 LLVKIRSGKK RTVEMHNKIY AIPIEIMQGT GEELFDHIVS CISDFLDYMG IKGPRMPLGF
601 TFSFPCQQTS LDAGILITWT KGFKATDCVG HDVVILLRDA IKRREEFDLD VVAVVNDTVG
661 TMMTCAYEEP TCEVGLIVGT GSNACYMEEM KNVEMVEGDQ GQMCINMEWG AFGDNGCLDD
721 IRTHYDRLVD EYSLNAGKQR YEKMISGMYL GEIVRNILID FTKKGFLFRG QISETLKTRG
781 IFETKELSQI ESDRLALLQV RAILQQLGLN STCDDSILVK TVCGVVSRRA AQLCGAGMAA
841 VVDKIRENRG LDRLNVTVGV DGTLYKLHPH FSRIMHQTVK ELSPKCNVSF LLSEDGSGKG
901 AALITAVGVR LRTEASS
(SEQ ID NO: 2)
    1 MDCEHSLSLP CRGAEAWEIG IDKYLYAMRL SDETLIDIMT RFRKEMKNGL SRDFNPTATV
  61 KMLPTFVRSI PDGSEKGDFI ALDLGGSSFR ILRVQVNHEK NQNVHMESEV YDTPENIVHG
121 SGSQLFDHVA ECLGDFMEKR KIKDKKLPVG FTFSFPCQQS KIDEAILITW TKREKASGVE
181 GADVVKLLNK AIKKRGDYDA NIVAVVNDTV GTMMTCGYDD QHCEVGLIIG TGTNACYMEE
241 LRHIDLVEGD EGRMCINTEW GAFGDDGSLE DIRTEFDREI DRGSLNPGKQ LFEKMVSGMY
301 LGELVRLILV KMAKEGLLFE GRITPELLTR GKENTSDVSA IEKNKEGLHN AKEILTRLGV
361 EPSDDDCVSV QHVCTIVSFR SANLVAATLG AILNRLRDNK GTPRLRTTVG VDGSLYKTHP
421 QYSRRFHKTL RRLVPDSDVR FLLSESGSGK GAAMVTAVAY RLAEQHRQIE ETLAHFHLTK
481 DMLLEVKKRM RAEMELGLRK QTHNNAVVKM LPSFVRRTPD GTENGDFLAL DLGGTNFRVL
541 LVKIRSGKKR TVEMHNKIYA IPIEIMQGTG EELFDHIVSC ISDFLDYMGI KGPRMPLGFT
601 FSFPCQQTSL DAGILITWTK GFKATDCVGH DVVTLLRDAI KRREEFDLDV VAVVNDTVGT
661 MMTCAYEEPT CEVGLIVGTG SNACYMEEMK NVEMVEGDQG QMCINMEWGA FGDNGCLDDI
721 RTHYDRLVDE YSLNAGKQRY EKMISGMYLG EIVRNILIDF TKKGFLFRGQ ISETLKTRGI
781 FETKFLSQIE SDRLALLQVR AILQQLGLNS TCDDSILVKT VCGVVSRRAA QLCGAGMAAV
841 VDKIRENRGL DRLNVTVGVD GTLYKLHPHF SRIMHQTVKE LSPKCNVSFL LSEDGSGKGA
901 ALITAVGVRL RTEASS
(SEQ ID NO: 3)
    1 MGQICQRESA TAAEKPKLHL LAESEIDKYL YAMRLSDETL IDIMTRFRKE MKNGLSRDFN
  61 PTATVKMLPT FVRSIPDGSE KGDFIALDLG GSSFRILRVQ VNHEKNQNVH MESEVYDTPE
121 NIVHGSGSQL FDHVAECLGD FMEKRKIKDK KLPVGFTFSF PCQQSKIDEA ILITWTKREK
181 ASGVEGADVV KLLNKAIKKR GDYDANIVAV VNDTVGTMMT CGYDDQHCEV GLIIGTGTNA
241 CYMEELRHID LVEGDEGRMC INTEWGAFGD DGSLEDIRTE FDREIDRGSL NPGKQLFEKM
301 VSGMYLGELV RLILVKMAKE GLLFEGRITP ELLTRGKFNT SDVSAIEKNK EGLHNAKEIL
361 TRLGVEPSDD DCVSVQHVCT IVSFRSANLV AATLGAILNR LRDNKGTPRL RTTVGVDGSL
421 YKTHPQYSRR FHKTLRRLVP DSDVRFLLSE SGSGKGAAMV TAVAYRLAEQ HRQIEETLAH
481 FHLTKDMLLE VKKRMRAEME LGLRKQTHNN AVVKMLPSFV RRTPDGTENG DFLALDLGGT
541 NFRVLLVKIR SGKKRTVEMH NKIYAIPIEI MQGTGEELFD HIVSCISDFL DYMGIKGPRM
601 PLGFTFSFPC QQTSLDAGIL ITWTRGFKAT DCVGHDVVTL LRDAIKRREE FDLDVVAVVN
661 DTVGTMMTCA YEEPTCEVGL IVGTGSNACY MEEMKNVEMV EGDQGQMCIN MEWGAFGDNG
721 CLDDIRTHYD RLVDEYSLNA GKQRYEKMIS GMYLGEIVRN ILIDFTKKGF LFRGQISETL
781 KTRGIFETKF LSQIESDRLA LLQVRAILQQ LGLNSTCDDS ILVKTVCGVV SRRAAQLCGA
841 GMAAVVDKIR ENRGLDRLNV TVGVDGTLYK LHPHFSRIMH QTVKELSPKC NVSELLSEDG
901 SGKGAALITA VGVRLRTEAS S
(SEQ ID NO: 4)
    1 MAKRALRDFI DKYLYAMRLS DETLIDIMTR FRKEMKNGLS RDFNPTATVK MLPTEVRSIP
  61 DGSEKGDF [symbol] A LDLGGSSER [symbol]  LRVQVNHEKN QNVHMESEVY DTPEN [symbol] VHGS GSQLFDHVAE
121 CLGDFMEKRK IKDKKLPVGF TFSFPCQQSK IDEAILITWT KRFKASGVEG ADVVKLLNKA
181 IKKRGDYDAN IVAVVNDTVG TMMTCGYDDQ HCEVGLIIGT GTNACYMEEL RHIDLVEGDE
241 GRMCINTEWG AFGDDGSLED IRTEFDREID RGSLNPGKQL FEKMVSGMYL GELVRLILVK
301 MAKEGLLFEG RITPELLTRG KENTSDVSAI EKNKEGLHNA KEILTRIGVE PSDDDCVSVQ
361 HVCTIVSFRS ANLVAATLGA ILNRLRDNKG TPRLRTTVGV DGSLYKTHPQ YSRRFHKTLR
421 RLVPDSDVRF LLSESGSGKG AAMVTAVAYR LAEQHRQIEE TLAHFHLTKD MLLEVKKRMR
481 AEMELGLRKQ THNNAVVKML PSFVRRTPDG TENGDFLALD LGGTNFRVLL VKIRSGKKRT
541 VEMHNKIYAI PIEIMQGTGE ELFDHIVSCI SDFLDYMGIK GPRMPLGFTF SFPCQQTSLD
601 AGILITWTKG FKATDCVGHD VVTLLRDAIK RREEFDLDVV AVVNDTVGTM MTCAYEEPTC
661 EVGLIVGTGS NACYMEEMKN VEMVEGDQGQ MCINMEWGAF GDNGCLDDIR THYDRLVDEY
721 SLNAGKQRYE KMISGMYLGE IVRNILIDFT KKGFLFRGQI SETLKTRGIF ETKFLSQIES
781 DRLALLQVRA ILQQLGLNST CDDSILVKTV CGVVSRRAAQ LCGAGMAAVV DKIRENRGLD
841 RLNVTVGVDG TLYKLHPHFS RIMHQTVKEL SPKCNVSFLL SEDGSGKGAA LITAVGVRLR
901 TEASS
```

[symbol]

indicates data missing or illegible when filed

As used herein, “hexokinase 2” or “hexokinase 2 isozyme” refers to any isoforms of hexokinase 2 and its naturally known variants, including that provided in SEQ ID NO: 5 as follows:

```
(SEQ ID NO: 5)
    1 MIASHLLAYF FTELNHDQVQ KVDQYLYHMR LSDETLLEIS KRFRKEMEKG LGATTHPTAA
  61 VKMLPTFVRS TPDGTEHGEF LALDLGGTNF RVLWVKVTDN GLQKVEMENQ IYAIPEDIMR
121 GSGTQLFDHI AECLANEMDK LQIKDKKLPL GFTFSFPCHQ TKLDESFLVS WTKGFKSSGV
181 EGRDVVAL [symbol] R KA [symbol] QRRGDFD [symbol]  D [symbol] VAVVNDT VGTMMTCGYD DHNCE [symbol] GL [symbol] V GTGSNACYME
241 EMRHIDMVEG DEGRMCINME WGAFGDDGSL NDIRTEFDQE IDMGSLNPGK QLFEKMISGM
301 YMGELVRLIL VKMAKEELLF GGKLSPELLN TGRFETKDIS DIEGEKDGIR KAREVLMREG
361 LDPTQEDCVA THRICQIVST RSASLCAATL AAVLQRIKEN KGEERLRSTI GVDGSVYKKH
421 PHFAKRLHKT VRRLVPGCDV RFLRSEDGSG KGAAMVTAVA YRLADQHRAR QKTLEHLQLS
481 HDQLLEVKRR MKVEMERGLS KETHASAPVK MLPTYVCATP DGTEKGDFLA LDLGGTNFRV
541 LLVRVRNGKW GGVEMHNKIY AIPQEVMHGT GDELFDHIVQ CIADFLEYMG MKGVSLPLGF
601 TFSFPCQQNS LDESILLKWT KGFKASGCEG EDVVTLLKEA IHRREEFDLD VVAVVNDTVG
661 TMMTCGFEDP HCEVGLIVGT GSNACYMEEM RNVELVEGEE GRMCVNMEWG AFGDNGCLDD
721 FRTEFDVAVD ELSLNPGKQR FEKMISGMYL GEIVRNILID FTKRGLLFRG RISERLKTRG
781 IFETFLSQI ESDCLALLQV RAILQHLGLE STCDDSIIVK EVCTVVARRA AQLCGAGMAA
841 VVDRIRENRG LDALKVTVGV DGTLYKLHPH FAKVMHETVK DLAPKCDVSF LQSEDGSGKG
901 AALITAVACR IREAGQR
```

[symbol]

indicates data missing or illegible when filed

In some examples, the 3-BP compositions described herein can further comprise a hexokinase inhibitor. The hexokinase inhibitor can be any molecule that inhibits hexokinase 1, hexokinase 2, and/or any isozyme thereof (collectively referred to herein as “hexokinase”).

As has been described, a major source of ATP production occurs in mitochondria in normal cells. However, ATP production from glycolysis is significantly upregulated in cancer cells. One reason for this upregulation is due to hexokinase molecules binding to, and forming complexes with, mitochondrial voltage dependent anion channels (VDACs) at ATP synthasomes, thus forming so called “ATP synthasome mega complexes.” The formation of such ATP synthasome mega complexes can immortalize the cancer cell, thus allowing the continued use of the cell's energy production processes for cancer growth. A hexokinase inhibitor, therefore, can thus block hexokinase from binding to the VADCs or displace hexokinase molecules from the VADCs of already formed ATP synthasome mega complexes.

In one example, a hexokinase inhibitor can be up to 25 amino acid units from the N-terminal region of Hexokinase 2 isozyme or Hexokinase 1 isozyme. In another example, the hexokinase inhibitor can be an amino acid sequence of 5 to 20 amino acid units, where the 5 to 20 amino acid sequence is present in the first 25 amino acid unit region beginning from the N-terminal end of hexokinase 1 isozyme or hexokinase 2 isozyme. In one example, the 5 to 20 amino acid sequence can be any 5-20 amino acid sequence present in the first 25 amino acid unit region of the N-terminus of Hexokinase 1 1 or Hexokinase 2. Such amino acid sequences can displace cellular bound hexokinase or competitively bind to voltage dependent anion channels (VDAC), thus preventing initial hexokinase binding.

In other examples, a hexokinase inhibitor can include antibodies against a portion of HK1 or HK2, such as, for example, the N-terminal region of either molecule. In one specific example, a hexokinase inhibitor can be an amino acid sequence, such as SEQ ID NO: 6, corresponding to the first 25 amino acids from the N-terminus end of hexokinase 1 (isoform 1) having a sequence as follows:

(SEQ ID NO: 6)
1 MIAAQLLAYY FTELKDDQVK KIDKY

In another example, a hexokinase inhibitor can be an amino acid sequence as in SEQ ID NO: 7, corresponding to the first 25 amino acids from the N-terminus end of hexokinase 1 (isoform 2) having a sequence as follows:

(SEQ ID NO: 7)
1 MDCEHSLSLP CRGARAWEIG IDKYL

In yet another example, a hexokinase inhibitor can be an amino acid sequence as in SEQ ID NO: 8, corresponding to the first 25 amino acids from the N-terminus end of hexokinase 1 (isoform 3) having a sequence as follows:

(SEQ ID NO: 8)
1 MGQICORESA TAAEKPKLHL LAESE

In still another example, a hexokinase inhibitor can be an amino acid sequence as in SEQ ID NO: 9, corresponding to the first 25 amino acids from the N-terminus end of hexokinase 1 (isoform 4) having a sequence as follows:

(SEQ ID NO: 9)
1 MAKRALRDFI DKYLYAMRLS DETLI

In yet another example, a hexokinase inhibitor can be an amino acid sequence as in SEQ ID NO: 10, corresponding to the first 25 amino acids from the N-terminus end of hexokinase 2 having a sequence as follows:

(SEQ ID NO: 10)
MIASHLLAYF FTELNHDQVQ KVDQY

Additional hexokinase inhibitors can be those as disclosed in U.S. Pat. No. 5,854,067 (to Newgard et al, issued Dec. 29, 1998) and/or U.S. Pat. No. 5,891,717 (to Newgard et al., issued Apr. 6, 1999), both of which are incorporated by reference in their entireties. Additional hexokinase inhibitors that can be used in the present formulations include those disclosed in U.S. Pat. Nos. 6,670,330; 6,218,435; 5,824,665; 5,652,273; and 5,643,883; and U.S. patent application publication Nos. 20030072814; 20020077300; and 20020035071; each of the foregoing patent publications and patent application is incorporated herein by reference, in their entireties.

In some examples, the 3-BP compositions described herein can further comprise various ingredients recited below. In the various dosage forms described above in FIGS. 4-7, any of these various ingredients can be admixed with the 3-BP, provided they are nonreactive therewith, or be present in a separate layer or in any layer described above, provided the ingredient(s) is/are reactively isolated in the storage form.

In one embodiment, the present compositions can include less biologically active amino acids as compared to their isomers to facilitate cancer cell starvation. In one aspect, the less biologically active amino acid can be a D-amino acid. However, if the L-amino acid is less biologically active than the D-form, the L-amino acid can be used.

In one embodiment, the present compositions can include inhibitors for DNA replication; inhibitors for DNA binding; and/or inhibitors for DNA transcription. In another embodiment, the present compositions can include inhibitors for cell cycle, growth and/or proliferation. In yet another embodiment, the present compositions can include inhibitors for signal transduction pathways. In yet another embodiment, the present compositions can include inhibitors for angiogenesis. In yet another embodiment, the present compositions can include small RNAs that interfere with normal gene control including antisense RNA, micro RNA, small hairpin RNA, short hairpin RNA, small interfering RNA, and the like. In yet another embodiment, the present compositions can include vitamin C; nutritional supplements including vitamins, CoQ10, flavonoids, free fatty acid, alpha lipoic acid, acai, gogi, mango, pomergrante, L-carnitine, selenium; etc.

In addition to the active agent(s), the storage form of the composition can also include a pharmaceutically acceptable carrier. The carrier can be a single composition, or a mixture of compositions. Additionally, the carrier can take the form of an encapsulation coat, an absorbing agent, a coating substance, a controlled release device, a release modifying agent, surfactants, or a combination thereof. In some aspects, the carrier can comprise about 0.01 wt % to about 99 wt % of the total composition. In one embodiment, the carrier can comprise about 1 wt % to about 95 wt % of the total formulation. In another embodiment, the carrier can comprise about 5 wt % to about 80 wt %. In yet a further embodiment, the carrier can comprise about 10 wt % to about 60 wt %. In one embodiment, the carrier can be admixed with the active agent(s). In another embodiment, the carrier can adsorb, entrap, or encapsulate at least a portion of the active agent(s).

Non-limiting examples of compounds that can be used as at least a part of the carrier include without limitation: cetyl alcohol and its esters; stearic acid and its glycerol esters, polyoxyethylene alkyl ethers; polyethylene glycol; polyglycolyzed glycerides; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; proteins; polyoxyethylene glycerides; polyoxyethylene sterols, derivatives, and analogues thereof; polyoxyethylene hydrogenated vegetable oils; reaction mixtures of polyols with at least one member of the group consisting of fatty acids, glycerides, vegetable oils, hydrogenated vegetable oils, and sterols; tocopherol derivatives, sugar esters; sugar ethers; sucroglycerides; waxes, shellac, pharmaceutically acceptable salts thereof, and mixtures thereof.

Non-limiting examples of release modifying agents include without limitation: polyethylene glycols having a weight average molecular weight of about 1000 and more, carbomer, methyl methacrylate copolymers, methacrylate copolymers, hydroxypropyl methyl cellulose, hydroxypropyl cellulose, cellulose acetate phthalate, ethyl cellulose, methyl cellulose and their derivatives; ion-exchange resin; mono-, di-, tri-esters of fatty acids with glycerol; tocopherol and its esters; sucrose esters with fatty acids; polyvinyl pyrollidone; xanthan gums; cetyl alcohol; waxes; fats and oils, proteins, alginate, polyvinyl polymers, gelatins, organic acids, and their derivatives and combinations thereof.

In one embodiment, the carrier can include at least one of celluloses; carbomers; methacrylates; dextrins; gums; inorganic carbonates or salts of calcium or magnesium or both; fatty acid esters; gelatin; lactoses; maltoses; mono-, di- or triglycerides; oils, polyethylene glycols; polyethylene oxide co-polymers; proteins; resins; shellac; silicates; starches; sugar stearates; partially or fully hydrogenated vegetable oils; waxes; and combinations thereof.

In yet another embodiment, the carrier can include at least one of celluloses; carbomers; methacrylates; inorganic carbonates or salts of calcium; inorganic carbonates or salts of magnesium; fatty acids; fatty acid esters; gelatin; lactoses; polyethylene glycol; polyethylene oxide co-polymers; silicates; partially or fully hydrogenated vegetable oils, and combinations thereof.

In yet a further embodiment, the carrier can include at least one of microcrystalline cellulose; hydroxypropyl methylcellulose; ethyl cellulose; silicon dioxide; colloidal silicon dioxide; magnesium aluminosilicate; lactose; xanthan gum; stearic acid; glyceryl distearate; hydrogenated vegetable oil; and combinations thereof.

In another example, various additives can be included in the 3-BP formulations of the present disclosure, including, without limitation: fillers such as lactoses, starches, sugars, celluloses, calcium salts, silicon oxides, metallosilicates and the like; disintegrants such as starch glycolate, lauryl sulfate, pregaltinized starch, croscarmellose, crospovidone and the like; binders such as pyrrolidones, methacrylates, vinyl acetates, gums, acacia; tragacanth; kaolins; carrageenan alginates, gelatins and the like; cosolvents such as alcohols, polyethylene glycols having average molecular weight of less than 1000, propylene glycols and the like; surface tension modifiers such as hydrophilic or amphiphlic surfactants; taste-masking agents; sweeteners; microencapsulating agents; process aids such as lubricants, glidants, talc, stearates, lecithin and the like; polymeric coating agents; plasticizers; buffers; organic acids; antioxidants; flavors; colors; alkalizers; humectants; sorbitols; mannitols; osmotic salts; proteins; resins; moisture repelling agents; hygroscopic agents; desiccants; and combinations thereof.

### EXAMPLES

The following examples pertain to specific embodiments and point out specific features, elements, or steps that can be used or otherwise combined in achieving such embodiments.

In one example, a liquid dispersible solid formulation can include an active agent dispersed in a pharmaceutically acceptable carrier, a reactive ingredient, and a reactivity isolation barrier disposed between the active agent and the reactive ingredient to preclude chemical contact therebetween, such that the active agent is stabilized.

In one example, the reactivity isolation barrier is a reactivity isolation barrier layer surrounding the active agent.

In one example, the reactivity isolation barrier layer is a reactivity isolation barrier coating surrounding the active agent.

In one example, the reactive ingredient is a reactive ingredient coating surrounding the reactivity isolation barrier coating.

In one example, liquid dispersible formulation further includes a disintegrable protective coating surrounding the reactive ingredient coating.

In one example, the reactive ingredient is reactive ingredient powder surrounding the reactivity isolation barrier coating.

In one example, the active agent is a plurality of active agent particulates, each including a reactivity isolation barrier coating, wherein the plurality of active agent particulates is dispersed in the reactive ingredient powder.

In one example, the active agent is encapsulated withing the reactivity isolation barrier layer.

In one example, the reactive ingredient is a biological buffer selected from the group consisting of a citrate, a succinate, a malate, an edetate, a histidine, an acetate, an adipate, an aconitate, an ascorbate, a benzoate, a carbonate, a bicarbonate, a maleate, a glutamate, a phosphate, a tartrate, and a combination thereof.

In one example, the biological buffer is a component selected from the group consisting of a citrate, an acetate, phosphate, and a combination thereof.

In one example, the reactivity isolation barrier includes a disintegrant selected from the group consisting of starches, sodium starch glycolates, clays, celluloses, methylcelluloses, carboxymethylcelluloses, alginates, pregelatinized corn starches, crospovidone, gums, and combinations thereof.

In one example, an anti-cancer liquid dispersible formulation can include a cellular energy inhibitor dispersed in a pharmaceutically acceptable carrier, a reactive ingredient, a reactivity isolation barrier disposed between the active agent and the reactive ingredient to preclude chemical contact therebetween, such that the active agent is stabilized, wherein the cellular energy inhibitor has the structure according to formula I

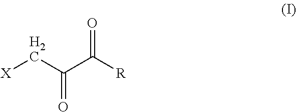

wherein X is selected from the group consisting of: a nitro, an imidazole, a halide, sulfonate, a carboxylate, an alkoxide, and amine oxide; and R is selected from the group consisting of: OR′, N(R″)₂, C(O)R″′, C1-C6 alkyl, C6-C12 aryl, C1-C6 heteroalkyl, a C6-C12 heteroaryl, H, and an alkali metal; where R′ represents H, alkali metal, C1-C6 alkyl, C6-C12 aryl or C(O)R′″, R″ represents H, C1-C6 alkyl, or C6-C12 aryl, and R″ represents H, C1-C20 alkyl or C6-C12 aryl.

In one example, the liquid dispersible formulation can further include at least one sugar, which stabilizes the cellular energy inhibitor by substantially preventing the inhibitor from hydrolyzing.

In one example, the reactive ingredient is a biological buffer that is present in an amount sufficient to at least partially deacidify the cellular energy inhibitor and neutralize metabolic by-products of the cellular energy inhibitor.

In one example, the cellular energy inhibitor is a 3-halopyruvate selected from 3-fluoropyruvate, 3-chloropyruvate, 3-bromopyruvate, 3-iodopyruvate, and combinations thereof.

In one example, the cellular energy inhibitor is 3-bromopyruvate.

In one example, the at least one sugar can be selected from gluconic acid, glucuronic acid, mannitol, erythritol, isomalt, lactitol, maltitol, sorbitol, xylitol, dulcitol, ribitol, inositol, glycerol, ethylene glycol, threitol, arabitol, galactitol, fucitol, iditol, volemitol, maltotriitol, maltotetraitol, polyglycitol, or a combination thereof.

In one example, the at least one sugar can be a five-carbon sugar.

In one example, the at least one sugar can be at least two five-carbon sugars.

In one example, the composition can include a second sugar selected from mannitol, erytritol, isomalt, lactitol, maltitol, sorbitol, xyolitol, dulcitol, ribitol, inositol, sorbitol, and combinations thereof.

In one example, the composition can include a second sugar and a third sugar independently selected from mannitol, erytritol, isomalt, lactitol, maltitol, sorbitol, xyolitol, dulcitol, ribitol, inositol, sorbitol, and combinations thereof.

In one example, the at least one sugar can include glycerol, inositol, and sorbitol.

In one example, the composition includes glycerol in a range from about 0.1 wt % to about 3 wt %, inositol in a range from about 1 wt % to about 5 wt %, and sorbitol in a range from about 30 wt % to about 50 wt %.

In one example, the composition can include d-lactic acid and epinephrine.

In one example, the composition can include a glycolysis inhibitor and wherein the glycolysis inhibitor is 2-deoxglucose in a concentration from about 1 mM to about 5 mM.

In one example, the composition can include the glycolysis inhibitor 2-deoxglucose.

In one example, the composition can include the 2-deoxglucose in a concentration from about 1 mM to about 5 mM.

In one example, the biological buffer is selected from one or more of a citrate buffer, a phosphate buffer, and an acetate buffer.

In one example, the biological buffer is a citrate buffer.

In one example, the biological buffer is a phosphate buffer.

In one example, the composition can include at least one additive selected from phospholipids; liposomes; nanoparticles; immune system modulators and/or immune system boosters including brown rice extract, muramyl dipeptide including analogues, mushroom extract, bioflavonoids, Vitamin D3-Binding Protein-Derived Macrophage Activating Factor (GcMAF), inhibitors of nagalase, threonine attached to N-acetylgalactosamine, and antibodies against nagalase; L-lactate dehydrogenase; D-lactate dehydrogenase; nicotinamide adenine dinucleotides; inhibitors for DNA replication; inhibitors for DNA binding; inhibitors for DNA transcription; inhibitors for cell cycle, growth and/or proliferation; inhibitors for signal transduction pathways; inhibitors for angiogensis; small RNAs that interfere with normal gene control interfering RNA; vitamin C; nutritional supplements including vitamins, CoQ10, flavonoids, free fatty acid, alpha lipoic acid, acai, gogi, mango, pomergrante, L-carnitine, selenium; a less biologically active amino acid as compared to its isomer; and mixtures thereof.

In one example, the composition can include a hexokinase inhibitor.

In one example, the hexokinase inhibitor inhibits binding of hexokinase 1 and/or hexokinase 2 to VDAC.

In one example, the hexokinase inhibitor is an amino acid sequence selected from the group consisting of: SEQ ID NO: 6, SEQ ID NO: 7, SEQ ID NO: 8, SEQ ID NO: 9, and SEQ ID NO. 10.

In one example, the composition can include a mitochondrial inhibitor.

In one example, the mitochondrial inhibitor is selected from oligomycin, efrapeptin, aurovertin, and mixtures thereof; in a concentration from about 0.01 mM to about 0.5 mM.

In one example, the mitochondrial inhibitor is in a concentration from about 0.01 mM to about 0.5 mM.

In one example, the reactivity isolation barrier is a reactivity isolation barrier layer surrounding the 3-bromopyruvate.

In one example, the reactivity isolation barrier layer is a reactivity isolation barrier coating surrounding the 3-bromopyruvate.

In one example, the reactive ingredient is a biological buffer coating surrounding the reactivity isolation barrier coating.

In one example, liquid dispersible formulation further includes a disintegrable protective coating surrounding the biological buffer coating.

In one example, the biological buffer is a biological buffer powder surrounding the reactivity isolation barrier coating.

In one example, the 3-bromopyruvate is a plurality of 3-bromopyruvate particulates, each including a reactivity isolation barrier coating, wherein the plurality of 3-bromopyruvate particulates is dispersed in the biological buffer powder.

In one example, the 3-bromopyruvate is encapsulated withing the reactivity isolation barrier layer.

In one example, the reactivity isolation barrier includes a disintegrant selected from the group consisting of starches, sodium starch glycolates, clays, celluloses, methylcelluloses, carboxymethylcelluloses, alginates, pregelatinized corn starches, crospovidone, gums, and combinations thereof.

In one example, an anti-cancer liquid dispersible formulation can include a cellular energy inhibitor admixed with a reactive ingredient dispersed in a pharmaceutically acceptable carrier, wherein the cellular energy inhibitor has the structure according to formula I

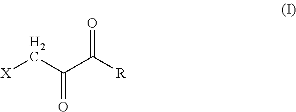

wherein X is selected from the group consisting of: a nitro, an imidazole, a halide, sulfonate, a carboxylate, an alkoxide, and amine oxide; and R is selected from the group consisting of: OR′, N(R″)₂, C(O)R″′, C1-C6 alkyl, C6-C12 aryl, C1-C6 heteroalkyl, a C6-C12 heteroaryl, H, and an alkali metal; where R′ represents H, alkali metal, C1-C6 alkyl, C6-C12 aryl or C(O)R′″, R″ represents H, C1-C6 alkyl, or C6-C12 aryl, and R″ represents H, C1-C20 alkyl or C6-C12 aryl.

In one example, the liquid dispersible formulation can further include at least one sugar, which stabilizes the cellular energy inhibitor by substantially preventing the inhibitor from hydrolyzing.

In one example, the reactive ingredient is a biological buffer that is present in an amount sufficient to at least partially deacidify the cellular energy inhibitor and neutralize metabolic by-products of the cellular energy inhibitor.

In one example, the cellular energy inhibitor is a 3-halopyruvate selected from 3-fluoropyruvate, 3-chloropyruvate, 3-bromopyruvate, 3-iodopyruvate, and combinations thereof.

In one example, the cellular energy inhibitor is 3-bromopyruvate.

In one example, the at least one sugar can be selected from gluconic acid, glucuronic acid, mannitol, erythritol, isomalt, lactitol, maltitol, sorbitol, xylitol, dulcitol, ribitol, inositol, myo inositol, glycerol, ethylene glycol, threitol, arabitol, galactitol, fucitol, iditol, volemitol, maltotriitol, maltotetraitol, polyglycitol, or a combination thereof.

In one example, the at least one sugar can be a five-carbon sugar.

In one example, the at least one sugar can be at least two five-carbon sugars.

In one example, the composition can include a second sugar selected from mannitol, erytritol, isomalt, lactitol, maltitol, sorbitol, xyolitol, dulcitol, ribitol, inositol, myo inositol, sorbitol, and combinations thereof.

In one example, the composition can include a second sugar and a third sugar independently selected from mannitol, erytritol, isomalt, lactitol, maltitol, sorbitol, xyolitol, dulcitol, ribitol, inositol, myo inositol, sorbitol, and combinations thereof.

In one example, the at least one sugar can include glycerol, myo inositol, and sorbitol.

In one example, the composition can include one or more sugars in a range from about 0.5 wt % to about 50.0 wt % or from about 1.0 wt % to about 25.5 wt %. In yet another example, a composition can include one or more sugars in a range from about 0.2 wt % to about 75.0 wt % or from about 0.5 wt % to about 50.0 wt %. In a further example, a composition can include one or more sugars in a range from about 0.1 wt % to about 25.0 wt %, from about 0.2 wt % to about 10.0 wt %.

In some examples, the composition can include glycerol in a range from about 0.1 wt % to about 5.0 wt % or from about 0.1 wt % to about 3.0 wt %. In other examples, the composition can include inositol in a range from about 0.1 wt % to about 10 wt %, from about 0.1 wt % to about 6 wt %. In further examples, the composition can include sorbitol in a range from about 0.1 wt % to about 40.0 wt % or from about 0.1 wt % to about 30 wt %. In yet further examples, the composition can include mannitol in a range from about 0.1 wt % to about 30 wt % or from about 0.1 wt % to about 10 wt %. Additionally, each of the sugars may be added in a volume up to a maximum solubility of the sugar in the formulation or composition.

In one example, the composition can include d-lactic acid and epinephrine.

In one example, the composition can include a glycolysis inhibitor and wherein the glycolysis inhibitor is 2-deoxglucose in a concentration from about 1 mM to about 5 mM.

In one example, the composition can include the glycolysis inhibitor 2-deoxglucose.

In one example, the composition can include the 2-deoxglucose in a concentration from about 1 mM to about 5 mM.

In one example, the biological buffer is selected from one or more of a citrate buffer, a phosphate buffer, and an acetate buffer.

In one example, the biological buffer is a citrate buffer.

In one example, the biological buffer is a phosphate buffer.

In one example, the composition can include at least one additive selected from phospholipids; liposomes; nanoparticles; immune system modulators and/or immune system boosters including brown rice extract, muramyl dipeptide including analogues, mushroom extract, bioflavonoids, Vitamin D3-Binding Protein-Derived Macrophage Activating Factor (GcMAF), inhibitors of nagalase, threonine attached to N-acetylgalactosamine, and antibodies against nagalase; L-lactate dehydrogenase; D-lactate dehydrogenase; nicotinamide adenine dinucleotides; inhibitors for DNA replication; inhibitors for DNA binding; inhibitors for DNA transcription; inhibitors for cell cycle, growth and/or proliferation; inhibitors for signal transduction pathways; inhibitors for angiogensis; small RNAs that interfere with normal gene control including antisense RNA, micro RNA, small hairpin RNA, short hairpin RNA, small interfering RNA; vitamin C; nutritional supplements including vitamins, CoQ10, flavonoids, free fatty acid, alpha lipoic acid, acai, gogi, mango, pomergrante, L-carnitine, selenium; a less biologically active amino acid as compared to its isomer; and mixtures thereof.

In one example, the composition can include a hexokinase inhibitor.

In one example, the hexokinase inhibitor inhibits binding of hexokinase 1 and/or hexokinase 2 to VDAC.

In one example, the hexokinase inhibitor is an amino acid sequence selected from the group consisting of: SEQ ID NO: 6, SEQ ID NO: 7, SEQ ID NO: 8, SEQ ID NO: 9, and SEQ ID NO. 10.

In one example, the composition can include a mitochondrial inhibitor.

In one example, the mitochondrial inhibitor is selected from oligomycin, efrapeptin, aurovertin, and mixtures thereof; in a concentration from about 0.01 mM to about 0.5 mM.

In one example, the mitochondrial inhibitor is in a concentration from about 0.01 mM to about 0.5 mM.

In one example, liquid dispersible formulation further includes a disintegrable protective coating.

In one example, the 3-bromopyruvate is a plurality of 3-bromopyruvate particulates dispersed in a biological buffer powder.

What is claimed is:

1. An anti-cancer formulation, comprising:
a dry liquid dispersible composition of a cellular energy inhibitor admixed with a reactive ingredient and a pharmaceutically acceptable carrier, wherein the cellular energy inhibitor has a structure according to formula I 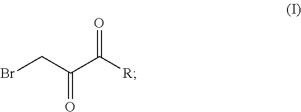
wherein R is selected from one of OR′, N(R″)₂, C(O)R″′, C1-C6 alkyl, C6-C12 aryl, C1-C6 heteroalkyl, C6-C12 heteroaryl, H, or an alkali metal,
where R′ is selected from one of H, an alkali metal, C1-C6 alkyl, C6-C12 aryl or C(O)R″′,
where R″ is selected from one of H, C1-C6 alkyl, or C6-C12 aryl,
and where R″′ is selected from on of H, C1-C20 alkyl or C6-C12 aryl.

2. The formulation of claim 1, wherein the cellular energy inhibitor has a structure according to formula II.

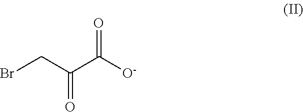

3. The formulation of claim 1, wherein the cellular energy inhibitor has a structure according to formula III.

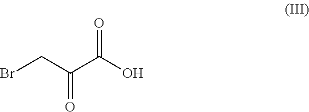

4. The formulation of claim 1, wherein the dry liquid dispersible composition further includes at least one sugar, which stabilizes the cellular energy inhibitor by substantially preventing the cellular energy inhibitor from hydrolyzing.

5. The formulation of claim 4, wherein the at least one sugar is selected from gluconic acid, glucuronic acid, mannitol, erythritol, isomalt, lactitol, maltitol, sorbitol, xylitol, dulcitol, ribitol, inositol, myo inositol, glycerol, ethylene glycol, threitol, arabitol, galactitol, fucitol, iditol, volemitol, maltotriitol, maltotetraitol, polyglycitol, or a combination thereof.

6. The formulation of claim 4, wherein the at least one sugar is a five-carbon sugar.

7. The formulation of claim 4, wherein the at least one sugar is at least two five-carbon sugars.

8. The formulation of claim 4, wherein the dry liquid dispersible composition further includes a second sugar selected from mannitol, erytritol, isomalt, lactitol, maltitol, sorbitol, xyolitol, dulcitol, ribitol, inositol, myo inositol, or sorbitol.

9. The formulation of claim 4, wherein the dry liquid dispersible composition further includes a second sugar and a third sugar independently selected from mannitol, erytritol, isomalt, lactitol, maltitol, sorbitol, xyolitol, dulcitol, ribitol, inositol, myo inositol, sorbitol, or a combination thereof.

10. The formulation of claim 4, wherein the at least one sugar includes glycerol, myo inositol, and sorbitol.

11. The formulation of claim 4, wherein the dry liquid dispersible composition includes glycerol in a range from about 0.1 wt % to about 5.0 wt % or from about 0.1 wt % to about 3.0 wt %.

12. The formulation of claim 4, wherein the dry liquid dispersible composition includes inositol in a range from about 0.1 wt % to about 10 wt % or from about 0.1 wt % to about 6 wt %.

13. The formulation of claim 4, wherein the dry liquid dispersible composition includes sorbitol in a range from about 0.1 wt % to about 40.0 wt % or from about 0.1 wt % to about 30 wt %.

14. The formulation of claim 4, wherein the dry liquid dispersible composition includes mannitol in a range from about 0.1 wt % to about 30 wt % or from about 0.1 wt % to about 10 wt %.

15. The formulation of claim 4, wherein the dry liquid dispersible composition includes the at least one sugar in a range from about 0.5 wt % to about 50.0 wt %, from about 1.0 wt % to about 25.5 wt %, from about 0.1 wt % to about 25.0 wt %, or from about 0.2 wt % to about 10.0 wt %.

16. The formulation of claim 1, wherein the reactive ingredient is a biological buffer in an amount sufficient to at least partially deacidify the cellular energy inhibitor and neutralize metabolic by-products of the cellular energy inhibitor.

17. The formulation of claim 1, wherein the biological buffer is selected from one or more of a citrate buffer, a phosphate buffer, or an acetate buffer.

18. The formulation of claim 1, further comprising 2-deoxglucose in a concentration from about 1 mM to about 5 mM.

19. The formulation of claim 1, further comprising at least one additive selected from phospholipids, liposomes, nanoparticles, immune system modulators and/or immune system boosters including brown rice extract, muramyl dipeptide including analogues, mushroom extracts, bioflavonoids, Vitamin D3-Binding Protein-Derived Macrophage Activating Factor (GcMAF), inhibitors of nagalase, threonine attached to N-acetylgalactosamine, and antibodies against nagalase, L-lactate dehydrogenase, D-lactate dehydrogenase, nicotinamide adenine dinucleotides, inhibitors for DNA replication, inhibitors for DNA binding, inhibitors for DNA transcription, inhibitors for cell cycle, growth and/or proliferation, inhibitors for signal transduction pathways, inhibitors for angiogenesis, small RNAs that interfere with normal gene control interfering RNA, vitamin C, nutritional supplements including vitamins, CoQ10, flavonoids, free fatty acid, alpha lipoic acid, acai, gogi, mango, pomergrante, L-carnitine, selenium, a less biologically active amino acid as compared to its isomer, or a combination thereof.

20. The formulation of claim 1, further comprising a hexokinase inhibitor that inhibits binding of hexokinase 1 and/or hexokinase 2 to VDAC.

21. The formulation of claim 20, wherein the hexokinase inhibitor has an amino acid sequence selected from SEQ ID NO. 6, SEQ ID NO. 7, SEQ ID NO. 8, SEQ ID NO. 9, and SEQ ID NO. 10.

22. The formulation of claim 1, further comprising a mitochondrial inhibitor in a concentration from about 0.01 mM to about 0.5 mM.

23. The formulation of claim 22, wherein the mitochondrial inhibitor is selected from oligomycin, efrapeptin, aurovertin, or a mixture thereof.

24. The formulation of claim 1, further comprising d-lactic acid and/or epinephrine.

25. The formulation of claim 1, wherein the dry liquid dispersible composition has a stability such that at least 90% w/w of 3-BP remains in the dry liquid dispersible composition after 24 weeks at −20° C.

26. A liquid dispersible solid formulation, comprising:
a pharmaceutically acceptable carrier;
a cellular energy inhibitor dispersed in the pharmaceutically acceptable carrier;
a reactive ingredient; and
a reactivity isolation barrier disposed between the cellular energy inhibitor and the reactive ingredient to preclude chemical contact therebetween, such that the cellular energy inhibitor is stabilized;
wherein the cellular energy inhibitor has a structure according to formula I 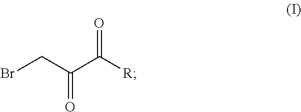
wherein R is selected from one of OR′, N(R″)₂, C(O)R″′, C1-C6 alkyl, C6-C12 aryl, C1-C6 heteroalkyl, C6-C12 heteroaryl, H, or an alkali metal,
where R′ is selected from one of H, an alkali metal, C1-C6 alkyl, C6-C12 aryl or C(O)R″′,
where R″ is selected from one of H, C1-C6 alkyl, or C6-C12 aryl,
and where R″′ is selected from on of H, C1-C20 alkyl or C6-C12 aryl.

27. The formulation of claim 26, wherein the reactivity isolation barrier is a coating surrounding the cellular energy inhibitor.

28. The formulation of claim 26, wherein the reactive ingredient is a coating surrounding the reactivity isolation barrier.

29. The formulation of claim 28, further comprising a disintegrable protective coating surrounding the reactive ingredient.

30. The formulation of claim 28, wherein the reactive ingredient is a powder surrounding the reactivity isolation barrier.

31. The formulation of claim 30, wherein the cellular energy inhibitor is a plurality of cellular energy inhibitor particulates, each coated with a reactivity isolation barrier, wherein the plurality of active cellular energy inhibitor particulates is dispersed in the reactive ingredient powder.

32. The formulation of claim 26, wherein the reactive ingredient is a biological buffer selected from a citrate, a succinate, a malate, an edetate, a histidine, an acetate, an adipate, an aconitate, an ascorbate, a benzoate, a carbonate, a bicarbonate, a maleate, a glutamate, a phosphate, a tartrate, or a combination thereof.

33. The formulation of claim 26, wherein the reactivity isolation barrier includes a disintegrant selected from starches, sodium starch glycolates, clays, celluloses, methylcelluloses, carboxymethylcelluloses, alginates, pregelatinized corn starches, crospovidone, gums, or a combination thereof.

34. The formulation of claim 26, further comprising at least one sugar, which stabilizes the cellular energy inhibitor by substantially preventing the cellular energy inhibitor from hydrolyzing.

35. The formulation of claim 34, wherein the at least one sugar is selected from gluconic acid, glucuronic acid, mannitol, erythritol, isomalt, lactitol, maltitol, sorbitol, xylitol, dulcitol, ribitol, inositol, myo inositol, glycerol, ethylene glycol, threitol, arabitol, galactitol, fucitol, iditol, volemitol, maltotriitol, maltotetraitol, polyglycitol, or a combination thereof.

36. The formulation of claim 34, wherein the at least one sugar is a five-carbon sugar.

37. The formulation of claim 34, wherein the at least one sugar is at least two five-carbon sugars.

38. The formulation of claim 33, further comprising a second sugar selected from mannitol, erytritol, isomalt, lactitol, maltitol, sorbitol, xyolitol, dulcitol, ribitol, inositol, myo inositol, or sorbitol.

39. The formulation of claim 34, further comprising a second sugar and a third sugar independently selected from mannitol, erytritol, isomalt, lactitol, maltitol, sorbitol, xyolitol, dulcitol, ribitol, inositol, myo inositol, sorbitol, or a combination thereof.

40. The formulation of claim 34, wherein the at least one sugar includes glycerol, myo inositol, and sorbitol.

41. The formulation of claim 34, further comprising glycerol in a range from about 0.1 wt % to about 5.0 wt % or from about 0.1 wt % to about 3.0 wt %.

42. The formulation of claim 34, further comprising inositol in a range from about 0.1 wt % to about 10 wt % or from about 0.1 wt % to about 6 wt %.

43. The formulation of claim 34, further comprising sorbitol in a range from about 0.1 wt % to about 40.0 wt % or from about 0.1 wt % to about 30 wt %.

44. The formulation of claim 34, further comprising mannitol in a range from about 0.1 wt % to about 30 wt % or from about 0.1 wt % to about 10 wt %.

45. The formulation of claim 34, further comprising the at least one sugar in a range from about 0.5 wt % to about 50.0 wt %, from about 1.0 wt % to about 25.5 wt %, from about 0.1 wt % to about 25.0 wt %, or from about 0.2 wt % to about 10.0 wt %.

46. The formulation of claim 26, wherein the reactive ingredient is a biological buffer in an amount sufficient to at least partially deacidify the cellular energy inhibitor and neutralize metabolic by-products of the cellular energy inhibitor.

47. The formulation of claim 26, wherein the biological buffer is selected from one or more of a citrate buffer, a phosphate buffer, or an acetate buffer.

48. The formulation of claim 26, further comprising 2-deoxglucose in a concentration from about 1 mM to about 5 mM.

49. The formulation of claim 26, further comprising at least one additive selected from phospholipids, liposomes, nanoparticles, immune system modulators and/or immune system boosters including brown rice extract, muramyl dipeptide including analogues, mushroom extracts, bioflavonoids, Vitamin D3-Binding Protein-Derived Macrophage Activating Factor (GcMAF), inhibitors of nagalase, threonine attached to N-acetylgalactosamine, and antibodies against nagalase, L-lactate dehydrogenase, D-lactate dehydrogenase, nicotinamide adenine dinucleotides, inhibitors for DNA replication, inhibitors for DNA binding, inhibitors for DNA transcription, inhibitors for cell cycle, growth and/or proliferation, inhibitors for signal transduction pathways, inhibitors for angiogenesis, small RNAs that interfere with normal gene control interfering RNA, vitamin C, nutritional supplements including vitamins, CoQ10, flavonoids, free fatty acid, alpha lipoic acid, acai, gogi, mango, pomergrante, L-carnitine, selenium, a less biologically active amino acid as compared to its isomer, or a combination thereof.

50. The formulation of claim 26, further comprising a hexokinase inhibitor that inhibits binding of hexokinase 1 and/or hexokinase 2 to VDAC.

51. The formulation of claim 50, wherein the hexokinase inhibitor has an amino acid sequence selected from SEQ ID NO. 6, SEQ ID NO. 7, SEQ ID NO. 8, SEQ ID NO. 9, and SEQ ID NO. 10.

52. The formulation of claim 26, further comprising a mitochondrial inhibitor in a concentration from about 0.01 mM to about 0.5 mM.

53. The formulation of claim 52, wherein the mitochondrial inhibitor is selected from oligomycin, efrapeptin, aurovertin, or a mixture thereof.

54. The formulation of claim 26, further comprising d-lactic acid and/or epinephrine.


SEQUENCE LISTING (10 sequences)

SEQ ID NO: 1 - protein, 917 aa, Homo sapiens
MIAAQLLAYYFTELKDDQVKKIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 2 - protein, 916 aa, Homo sapiens
MDCEHSLSLPCRGAEAWEIGIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 3 - protein, 921 aa, Homo sapiens
MGQICQRESATAAEKPKLHLLAESEIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 4 - protein, 905 aa, Homo sapiens
MAKRALRDFIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 5 - protein, 917 aa, Homo sapiens
MIASHLLAYFFTELNHDQVQKVDQYLYHMRLSDETLLEISKRFRKEMEKGLGATTHPTAAVKMLPTFVRSTPDGTEHGEFLALDLGGTNFRVLWVKVTDNGLQKVEMENQIYAIPEDIMRGSGTQLFDHIAECLANFMDKLQIKDKKLPLGFTFSFPCHQTKLDESFLVSWTKGFKSSGVEGRDVVALIRKAIQRRGDFDIDIVAVVNDTVGTMMTCGYDDHNCEIGLIVGTGSNACYMEEMRHIDMVEGDEGRMCINMEWGAFGDDGSLNDIRTEFDQEIDMGSLNPGKQLFEKMISGMYMGELVRLILVKMAKEELLFGGKLSPELLNTGRFETKDISDIEGEKDGIRKAREVLMRLGLDPTQEDCVATHRICQIVSTRSASLCAATLAAVLQRIKENKGEERLRSTIGVDGSVYKKHPHFAKRLHKTVRRLVPGCDVRFLRSEDGSGKGAAMVTAVAYRLADQHRARQKTLEHLQLSHDQLLEVKRRMKVEMERGLSKETHASAPVKMLPTYVCATPDGTEKGDFLALDLGGTNFRVLLVRVRNGKWGGVEMHNKIYAIPQEVMHGTGDELFDHIVQCIADFLEYMGMKGVSLPLGFTFSFPCQQNSLDESILLKWTKGFKASGCEGEDVVTLLKEAIHRREEFDLDVVAVVNDTVGTMMTCGFEDPHCEVGLIVGTGSNACYMEEMRNVELVEGEEGRMCVNMEWGAFGDNGCLDDFRTEFDVAVDELSLNPGKQRFEKMISGMYLGEIVRNILIDFTKRGLLFRGRISERLKTRGIFETKFLSQIESDCLALLQVRAILQHLGLESTCDDSIIVKEVCTVVARRAAQLCGAGMAAVVDRIRENRGLDALKVTVGVDGTLYKLHPHFAKVMHETVKDLAPKCDVSFLQSEDGSGKGAALITAVACRIREAGQR

SEQ ID NO: 6 - protein, 25 aa, Homo sapiens
MIAAQLLAYYFTELKDDQVKKIDKY

SEQ ID NO: 7 - protein, 25 aa, Homo sapiens
MDCEHSLSLPCRGAEAWEIGIDKYL

SEQ ID NO: 8 - protein, 25 aa, Homo sapiens
MGQICQRESATAAEKPKLHLLAESE

SEQ ID NO: 9 - protein, 25 aa, Homo sapiens
MAKRALRDFIDKYLYAMRLSDETLI

SEQ ID NO: 10 - protein, 25 aa, Homo sapiens
MIASHLLAYFFTELNHDQVQKVDQY